(19) United States
(12) Patent Application Publication
(10) Pub. No.: US 2022/0363767 A1
(43) Pub. Date: Nov. 17, 2022

(54) HLA-H, HLA-J, HLA-L, HLA-V AND HLA-Y AS THERAPEUTIC AND DIAGNOSTIC TARGETS

(71) Applicant: Intellexon GmbH, Pöcking (DE)

(72) Inventors: Wolfgang WÜRFEL, Odelzhausen (DE); Ralph Markus WIRTZ, Köln (DE); Christoph WINTERHALTER, Pöcking (DE); Franziska WÜRFEL, Odelzhausen (DE)

(21) Appl. No.: 17/771,793

(22) PCT Filed: Oct. 19, 2020
(86) PCT No.: PCT/EP2020/079344

§ 371 Date: Apr. 25, 2022

(30) Foreign Application Priority Data
Oct. 25, 2019 (EP) 19205451.8

(51) Int. Cl. C07K 16/28; C07K 14/74; C12Q 1/6881; C12Q 1/6886; G01N 33/574; G01N 33/60; G01N 33/68

(52) U.S. Cl. CPC C07K 16/2833; C07K 14/70539; C12Q 1/6881; C12Q 1/6886; G01N 33/57484; G01N 33/60; G01N 33/68; C12Q 2600/158

(57) ABSTRACT

The present invention relates to a method for producing a medicament for the treatment or prevention of a tumor in a subject or a diagnostic agent for the detection of a tumor in a subject comprising (A) determining the expression of at least one nucleic acid molecule and/or at least one protein or peptide in a sample obtained from said subject, wherein the at least one nucleic acid molecule is selected from nucleic acid molecules (a) encoding a polypeptide comprising or consisting of the amino acid sequence of any one of SEQ ID NOs 1 to 5, (b) comprising or consisting of the nucleotide sequence of any one of SEQ ID NOs 6 to 10, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and wherein the at least one protein or peptide is selected from proteins or peptides being encoded by the nucleic acid molecule of any one of (a) to (g); and (B) producing a medicament capable of inhibiting the expression of the at least nucleic acid molecule and/or the at least one protein or peptide in the subject, if the at least one nucleic acid molecule and/or at least one protein or peptide is expressed in (A), and/or (B′) producing a diagnostic agent capable of detecting in vivo the sites of expression of the at least nucleic acid molecule and/or the at least one protein or peptide in the subject, if the at least one nucleic acid molecule and/or at least one protein or peptide is expressed in (A).

The present invention relates to a method for producing a medicament for the treatment or prevention of a tumor in a subject or a diagnostic agent for the detection of a tumor in a subject comprising (A) determining the expression of at least one nucleic acid molecule and/or at least one protein or peptide in a sample obtained from said subject, wherein the at least one nucleic acid molecule is selected from nucleic acid molecules (a) encoding a polypeptide comprising or consisting of the amino acid sequence of any one of SEQ ID NOs 1 to 5, (b) comprising or consisting of the nucleotide sequence of any one of SEQ ID NOs 6 to 10, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and wherein the at least one protein or peptide is selected from proteins or peptides being encoded by the nucleic acid molecule of any one of (a) to (g); and (B) producing a medicament capable of inhibiting the expression of the at least nucleic acid molecule and/or the at least one protein or peptide in the subject, if the at least one nucleic acid molecule and/or at least one protein or peptide is expressed in (A), and/or (B′) producing a diagnostic agent capable of detecting in vivo the sites of expression of the at least nucleic acid molecule and/or the at least one protein or peptide in the subject, if the at least one nucleic acid molecule and/or at least one protein or peptide is expressed in (A).

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Personalized tumor therapy is a treatment strategy centered on the ability to predict which patients are more likely to respond to specific tumor therapies. This approach is based on the idea that tumor markers are associated with patient prognosis and tumor response to therapy. Tumor markers can be DNA, RNA, protein and metabolomic profiles that predict therapy response.

Selecting the right treatment for patients with tumor is a complex decision based on continuously evolving molecular diagnostics and rapidly emerging biomedical literature. Tracking associations between actionable genomic alterations and targeted therapies in clinical trials can be challenging for treating oncologists and researchers alike. Chemotherapy has remained the backbone of cancer treatment for many tumor types, but has limited response rates and notable side effects. The driver molecular mechanisms involved in cancer initiation, progression, and resistance are increasingly pursued as therapeutic targets. Examples of successful personalized tumor treatments have revolutionized oncology, such as targeting HER2 in breast, bcr-abl in chronic myeloid leukemia, or ALK in non-small-cell lung cancer (NSCLC).

Precision medicine in oncology spans a continuum, ranging from efforts to identify diagnostic biomarkers (to detect the occurrence of cancer in healthy patients, to identify tumors earlier), prognostic biomarkers (to predict the natural course of the disease), predictive biomarkers (to predict the clinical outcome in the presence of a specific therapy), and pharmacogenomic biomarkers (to identify alterations in drug metabolism and predict response and toxicities related to a specific treatment).

However, there is still an urgent need to identify new means and methods that can be used for the treatment of/in the valuation of tumors and as therapeutic and diagnostic targets in order to arrive at an effective personalized tumor therapy and tumor diagnosis. This need is addressed by the present invention.

Hence, the present invention relates in a first aspect to a method for producing a medicament for the treatment or prevention of a tumor in a subject or a diagnostic agent for the detection of a tumor in a subject comprising (A) determining the expression of at least one nucleic acid molecule and/or at least one protein or peptide in a sample obtained from said subject, wherein the at least one nucleic acid molecule is selected from nucleic acid molecules (a) encoding a polypeptide comprising or consisting of the amino acid sequence of any one of SEQ ID NOs 1 to 5, (b) comprising or consisting of the nucleotide sequence of any one of SEQ ID NOs 6 to 10, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and wherein the at least one protein or peptide is selected from proteins or peptides being encoded by the nucleic acid molecule of any one of (a) to (g); and (B) producing a medicament capable of inhibiting the expression of the at least nucleic acid molecule and/or the at least one protein or peptide in the subject, if the at least one nucleic acid molecule and/or at least one protein or peptide is expressed in (A), and/or (B′) producing a diagnostic agent capable of detecting in vivo the sites of expression of the at least nucleic acid molecule and/or the at least one protein or peptide in the subject, if the at least one nucleic acid molecule and/or at least one protein or peptide is expressed in (A).

The term “medicament” as used herein designates a compound or combination of compounds that is pharmaceutically active with respect to the treatment or prevention of a tumor. The term “diagnostic agent” as used herein designates a compound or combination of compounds that can be used for the detection of a tumor in a subject. For instance and as will be further detailed herein below, the diagnostic agent can be labelled with a detectable label which then allows to detect tumor lesions in vivo. A tumor lesion is an area of tumor tissue in a subject. Medicaments as well as diagnostic agents can be administered to a subject.

The nature of the medicament is not particularly limited as long as it is capable of inhibiting the expression of the at least one nucleic acid molecule and/or the at least one protein or peptide in the subject in accordance with the invention, if the at least one nucleic acid molecule and/or at least one protein or peptide is expressed in step (A) of the method of the first aspect of the invention. Similarly, the nature of the diagnostic agent is not particularly limited as long as it is capable of detecting in vivo the sites of expression of the at least one nucleic acid molecule and/or the at least one protein or peptide in the subject in accordance with the invention, if the at least one nucleic acid molecule and/or at least one protein or peptide is expressed in step (A) of the method of the first aspect of the invention. With respect to the sites of expression to is be understood that a tumor generally originates at a particular body site which is called the primary tumor site. As a result of tumor growth the tumor may form metastases at distinct sites in the body, at so-called secondary tumor sites. The diagnostic agent is preferably capable to at least detect the primary tumor site and more preferably both the primary and (at least in part) the secondary tumour sites, if present.

The medicament preferably specifically inhibits and the diagnostic agent preferably specifically detects the at least one nucleic acid molecule or the at least one protein or peptide in accordance with the invention. This means that the medicament does not or essentially does not inhibit other nucleic acid molecules or proteins or peptides. This also means that the diagnostic agent does not or essentially does not detect other nucleic acid molecules or proteins or peptides. In particular, it is preferred that no other HLA nucleic acid molecules or proteins or peptides than the respective selected target HLA nucleic acid molecules or proteins or peptides are inhibited or detected.

The term “subject” in accordance with the invention refers to a mammal, preferably a domestic animal or a pet animal such as horse, cattle, pig, sheep, goat, dog or cat, and most preferably a human. The subject may be a subject being suspected to have cancer or a subject known to have cancer. In the latter case the subject may already have received a cancer therapy which was not effective. It is also possible to use the method before and the after the therapy in order to determine whether the therapy has changed the expression.

A tumor is an abnormal benign or malignant new growth of tissue that possesses no physiological function and arises from uncontrolled usually rapid cellular proliferation. The tumor is preferably cancer. Cancer is an abnormal malignant new growth of tissue that possesses no physiological function and arises from uncontrolled usually rapid cellular proliferation. The cancer is preferably selected from the group consisting of breast cancer, ovarian cancer, vaginal cancer, vulva cancer, bladder cancer, salivary gland cancer, endometrium cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias. Preferred cancers will be defined herein below.

The tumor or cancer is preferably a solid tumor or cancer. A solid tumor or cancer is an abnormal mass of tissue that usually does not contain cysts or liquid areas by contrast to a non-solid tumor (e.g. leukemia).

The nucleic acid sequences of SEQ ID NOs 6 to 10 are the genes encoding human HLA-H, HLA-J, HLA-L, HLA-V and HLA-Y respectively. It is preferred that the nucleic acid molecule according to the invention is genomic DNA or mRNA. In the case of mRNA, the nucleic acid molecule may in addition comprise a poly-A tail.

The amino acid sequences of SEQ ID NOs 1 to 5 are the soluble human HLA proteins HLA-H, HLA-J, HLA-L, HLA-V and HLA-Y, respectively. The HLA proteins are soluble since they do not comprise a transmembrane domain.

With respect to HLA-L is of note that SEQ ID NO: 3 is and SEQ ID NO: 8 encodes the soluble form of HLA-L and that HLA-L can also be found in the membrane-bound form of SEQ ID NOs 11 (amino acid sequence) and 12 (nucleotide sequence). This membrane-bound form can be released from the membrane by proteolytic cleavage from the membrane. Such HLA forms which become soluble by detachment from the membrane are also known as shedded isoforms; see Rizzo et al. (2013), Mol Cell Biochem.; 381(1-2):243-55. Hence, the nucleic acid molecules derived from SEQ ID: 8 as defined in the first aspect of the invention, and the proteins or peptides derived from SEQ ID NO: 3 as defined in the first aspect of the invention may also be derived from the membrane-bound form of SEQ ID NOs 12 and 11, respectively. With respect to determining the expression of HLA-L or sequences derived thereof it is also preferred to confine the determination to the soluble forms, so that the membrane-bound from is not determined. This can be done, for example, by removing cells and cell membranes from the sample prior to analysis.

The term “nucleic acid sequence” or “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. Certain nucleic acid molecules, for example, shRNAs, miRNAs, or an antisense nucleic acid molecules as described herein below, may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule may additionally comprise promoters, enhancers, response elements, signal sequences, polyadenylation sequences, introns, 5′- and 3′-non-coding regions, and the like.

The term “protein” as used herein interchangeably with the term “polypeptide” describes linear molecular chains of amino acids, including single chain proteins or their fragments, containing at least 50 amino acids. The term “peptide” as used herein describes a group of molecules consisting of up to 49 amino acids. The term “peptide” as used herein describes a group of molecules consisting with increased preference of at least 15 amino acids, at least 20 amino acids at least 25 amino acids, and at least 40 amino acids. The group of peptides and polypeptides are referred to together by using the term “(poly)peptide”. (Poly)peptides may further form oligomers consisting of at least two identical or different molecules. The corresponding higher order structures of such multimers are, correspondingly, termed homo- or heterodimers, homo- or heterotrimers etc. For example, the HLA proteins comprise cysteins and thus potential dimerization sites. The terms “(poly)peptide” and “protein” also refer to naturally modified (poly)peptides and proteins where the modification is effected e.g. by glycosylation, acetylation, phosphorylation and similar modifications which are well known in the art.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment for two or more sequences or subsequences the percentage of amino acid residues or nucleotides that are the same (e.g. 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

As defined herein, sequence identities of at least 85% identity, preferably at least 90% identity, and most preferred at least 95% identity are envisaged by the invention. However, also envisaged by the invention are with increasing preference sequence identities of at least 97.5%, at least 98.5%, at least 99%, at least 99.5%, at least 99.8%, and 100% identity.

The term “degenerate” as used herein refers to the degeneracy of the genetic code. Degeneracy of codons is the redundancy of the genetic code, exhibited as the multiplicity of three-base pair codon combinations that specify an amino acid. The degeneracy of the genetic code is what accounts for the existence of synonymous mutations.

The sample may be a body fluid of the subject or a tissue sample from an organ of the subject. Non-limiting examples of body fluids are whole blood, blood plasma, blood serum, urine, peritoneal fluid, and pleural fluid, liquor cerebrospinalis, tear fluid, or cells therefrom in solution. Non-limiting examples of tissue are colon, liver, breast, ovary, and testis. Tissue samples may be taken by aspiration or punctuation, excision or by any other surgical method leading to biopsy or resected cellular material. The sample may be a processed sample, e.g. a sample which has been frozen, fixed, embedded or the like. A preferred type of sample is a formaline fixed paraffin embedded (FFPE) sample. Preparation of FFPE samples are standard medical practice and these samples can be conserved for long periods of time.

Methods for assessing the expression, preferably the expression level of the nucleic acid molecule or the protein or peptide in the context of the method of the invention are established in the art.

For instance, the expression of the nucleic acid molecule may be assessed by real time quantitative PCR (RT-qPCR), electrophoretic techniques or a DNA Microarray (Roth (2002), Curr. Issues Mol. Biol., 4: 93-100), wherein a RT-qPCR is preferred. In these methods the expression level may be normalized against the (mean) expression level of one or more reference genes in the sample. The term “reference gene”, as used herein, is meant to refer to a gene which has a relatively invariable level of expression on the RNA transcript/mRNA level in the system which is being examined, i.e. the tumor. Such a gene may be referred to as a housekeeping gene. Non-limiting examples of reference genes are CALM2, B2M, RPL37A, GUSB, HPRT1 and GAPDH, preferably CALM2 and/or B2M. Other suitable reference genes are known to a person skilled in the art.

RT-qPCR is carried out in a thermal cycler with the capacity to illuminate each sample with a beam of light of at least one specified wavelength and detect the fluorescence emitted by the excited fluorophore. The thermal cycler is also able to rapidly heat and chill samples, thereby taking advantage of the physicochemical properties of the nucleic acids and DNA polymerase. The two common methods for the detection of PCR products in real-time qPCR are: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence (e.g. a TaqMan probe). The probes are generally fluorescently labeled probes. Preferably, a fluorescently labeled probe consists of an oligonucleotide labeled with both a fluorescent reporter dye and a quencher dye (=dual-label probe). Suitable fluorescent reporter and quencher dyes/moieties are known to a person skilled in the art and include, but are not limited to the reporter dyes/moieties 6-FAM™, JOE™, Cy5®, Cy3® and the quencher dyes/moieties dabcyl, TAMRA™, BHQ™-1, -2 or -3. Preferably primers for use in accordance with the present invention have a length of 15 to 30 nucleotides, and are in particular deoxyribonucleotides. In one embodiment, the primers are designed so as to (1) be specific for the target mRNA-sequence of as HLA gene or being derived therefrom, (2) provide an amplicon size of less than 120 bp (preferably less than 100 bp), (3) be mRNA-specific (consideration of exons/introns; preferably no amplification of genomic DNA), (4) have no tendency to dimerize and/or (5) have a melting temperature Tₘ in the range of from 58° C. to 62° C. (preferably, Tₘ is approximately 60° C.). As mentioned, the probe is required for a RT-qPCR according to (2) but the probe can be replaced by an intercalating dye in the case of a RT-qPCR according to (1), such as SYBR green.

RT-qPCR is illustrated in the examples herein below. Specific primers for detecting the expression of HLA-H, J, L and G as disclosed in Table 1. One or more of these primer pairs are preferably used for the detection the expression of HLA-H, J, L and/or G or the nucleic acid molecules derived thereof as defined herein. Each of these primer pairs is more preferably used together with the respective probe as shown in Table 1.

As one alternative of qPCR also electrophoretic techniques or as one further alternative a DNA microarray may be used to obtaining the levels of the nucleic acid molecule of the first aspect of the invention. The conventional approach to mRNA identification and quantitation is through a combination of gel electrophoresis, which provides information on size, and sequence-specific probing. The Northern blot is the most commonly applied technique in this latter class. The ribonuclease protection assay (RPA) was developed as a more sensitive, less labor-intensive alternative to the Northern blot. Hybridization is performed with a labeled ribonucleotide probe in solution, after which non-hybridized sample and probe are digested with a mixture of ribonucleases (e.g., RNase A and RNase T1) that selectively degrade single-stranded RNAs. Subsequent denaturing polyacrylamide gel electrophoresis provides a means for quantitation and also gives the size of the region hybridized by the probe. For both Northern blot and RPA, the accuracy and precision of quantitation are functions of the detection method and the reference or standard utilized. Most commonly, the probes are radiolabeled with 32P or 33P, in which case the final gel is exposed to X-ray film or phosphor screen and the intensity of each band quantified with a densitometer or phosphor imager, respectively. In both cases, the exposure time can be adjusted to suit the sensitivity required, but the phosphor-based technique is generally more sensitive and has a greater dynamic range. As an alternative to using radioactivity, probes can be labeled with an antigen or hapten, which is subsequently bound by a horseradish peroxidase- or alkaline phosphatase-conjugated antibody and quantified after addition of substrate by chemiluminescence on film or a fluorescence imager. In all of these imaging applications, subtraction of the background from a neighboring region of the gel without probe should be performed. The great advantage of the gel format is that any reference standards can be imaged simultaneously with the sample. Likewise, detection of a housekeeping gene is performed under the same conditions for all samples.

In addition, next generation sequencing (NGS) may be used (Behjati and Tarpey, Arch Dis Child Educ Pract Ed. 2013 December; 98(6): 236). NGS is a RNA or DNA sequencing technology which has revolutionised genomic research. Using NGS an entire human genome can be sequenced within a single day. In contrast, the previous Sanger sequencing technology, used to decipher the human genome, required over a decade to deliver the final draft. In view of the present invention NGS could be used to quantify in open configuration (genome wide exome sequencing) or as focussed panel harbouring the respective HLA genes and isoforms disclosed in this application.

For the construction of DNA microarrays two technologies have emerged. Generally, the starting point in each case for the design of an array is a set of sequences corresponding to the genes or putative genes to be probed. In the first approach, oligonucleotide probes are synthesized chemically on a glass substrate. Because of the variable efficiency of oligonucleotide hybridization to cDNA probes, multiple oligonucleotide probes are synthesized complementary to each gene of interest. Furthermore, for each fully complementary oligonucleotide on the array, an oligonucleotide with a mismatch at a single nucleotide position is constructed and used for normalization. Oligonucleotide arrays are routinely created with densities of about 10⁴-10⁶ probes/cm². The second major technology for DNA microarray construction is the robotic printing of cDNA probes directly onto a glass slide or other suitable substrate. A DNA clone is obtained for each gene of interest, purified, and amplified from a common vector by PCR using universal primers. The probes are robotically deposited in spots on the order of 50-200 μm in size. At this spacing, a density of, for example, approximately 10³ probes/cm² can be achieved.

Expression of the protein or peptide may be determined, for example, by using a “molecule binding to the protein or peptide” and preferably a “molecule specifically binding to the protein or peptide”. A molecule binding to the protein or peptide designates a molecule which under known conditions occurs predominantly bound to the protein or peptide. Expression of the protein or peptide may also be obtained by using Western Blot analysis, mass spectrometry analysis, FACS-analysis, ELISA, and immunohistochemistry. These techniques are non-limiting examples of methods which may be used to qualitatively, semi-quantitatively and/or quantitatively detect a protein or peptide.

Western blot analysis is a widely used and well-know analytical technique used to detect specific proteins or peptides in a given sample, for example, a tissue homogenate or body extract. It uses gel electrophoresis to separate native or denatured proteins or peptides by the length of the (poly)peptide (denaturing conditions) or by the 3-D structure of the protein (native/ non-denaturing conditions). The proteins or peptides are then transferred to a membrane (typically nitrocellulose or PVDF), where they are probed (detected) using antibodies specific to the target protein.

Also mass spectrometry (MS) analysis is a widely used and well-know analytical technique, wherein the mass-to-charge ratio of charged particles is measured. Mass spectrometry is used for determining masses of particles, for determining the elemental composition of a sample or molecule, and for elucidating the chemical structures of molecules, such as proteins, peptides and other chemical compounds. The MS principle consists of ionizing chemical compounds to generate charged molecules or molecule fragments and measuring their mass-to-charge ratios.

Fluorescence activated cell sorting (FACS) analysis is a widely used and well-known analytical technique, wherein biological cells are sorted based upon the specific light scattering of the fluorescent characteristics of each cell. Cells may be fixed in 4% formaldehyde, permeabilized with 0.2% Triton-X-100, and incubated with a fluorophore-labeled antibody (e.g. mono- or polyclonal anti-HLA antibody).

Enzyme-linked immunosorbent assay (ELISA) also is a widely used and well-know sensitive analytical technique, wherein an enzyme is linked to an antibody or antigen as a marker for the detection of a specific protein or peptide.

Immunohistochemistry (IHC) is the most common application of immunostaining. It involves the process of selectively identifying antigens (proteins) in cells of a tissue section by exploiting the principle of antibodies binding specifically to antigens in biological tissues. In combination with particular devices IHC can be used for quantitative in situ assessment of protein expression (for review Cregger et al. (2006) Arch Pathol Lab Med, 130:1026-1030). Quantitative IHC takes advantage of the fact that staining intensity correlates with absolute protein levels.

It was previously surprisingly found by the applicant that HLA-L, HLA-H and HLA-J were erroneously annotated as pseudogenes in the art. In fact, these genes are protein-coding and the expression of HLA-L, HLA-H and HLA-J was detected in various cancers (PCT/EP2019/060606, EP 19 18 4729.2, EP 19 18 4681.5 and EP 19 18 4717.7). Moreover, a promoter region and an open reading frame was also found in HLA-V and HLA-Y. Since HLA-L, HLA-H, HLA-J, HLA-V and HLA-Y all were erroneously annotated in the art, HLA-L, HLA-H, HLA-J, HLA-V and HLA-Y may be collectively described as new HLA-group, which is called herein class Iw. In addition, high expression level of HLA-L, HLA-H and HLA-J in patients having bladder cancer was found to be adversely associated with the survival of these patients. The higher the expression level of these HLA genes the more likely the patients died from the cancer within 2 years (EP 19 18 4681.5 and EP 19 18 4717.7). This body of evidence shows that the expression of the soluble HLA forms L, H and J is used by tumors as a mechanism of evading the immune system of the tumor patient. The same can be assumed for HLA-V and HLA-Y. Without wishing to be bound by this theory the inventors believe that these soluble HLAs forms from a cloud around the tumor cells which prevents the tumor cells from being recognized by the immune system of the tumor patient. It follows that a medicament capable of inhibiting HLA-L, HLA-H, HLA-J, HLA-V and/or HLA-Y at the nucleic acid or protein level is a suitable means for the treatment and prevention of tumor. Likewise, a diagnostic agent being capable of detecting in vivo the sites of HLA-L, HLA-H, HLA-J, HLA-V and/or HLA-Y at the nucleic acid or protein level is capable of diagnosing a tumor in a subject. However, since tumor cells are heterogenous not each and every tumor may use the expression of one or more of soluble HLA-L, HLA-H, HLA-J, HLA-V and HLA-Y to escape the immune system. For this reason the method of the first aspect of the invention determines the expression of HLA-L, HLA-H, HLA-J, HLA-V and HLA-Y at the nucleic acid and/or protein level (step A) and then produces a medicament (step B) and/or diagnostic agent (B′), if HLA-L, HLA-H, HLA-J, HLA-V and/or HLA-Y is/are expressed. This medicament or diagnostic agent may then inter alia be used as a personally tailored medicament or diagnostic agent for the subject from which the sample has been obtained as being used in the method of the first aspect of the invention. This approach is further illustrated by the appended examples. Examples 1 and 2 show the imaging and detection of HLA expression. In Examples 3 and 4 the generation of anti-HLA diagnostic and therapeutic agents is shown. Example 5 relates to the diagnosis of HLA expression in cancer mouse model. Finally, Examples 6 to 9 illustrate the diagnosis of HLA expression as well as an anti-HLA therapy in cancer patients.

In accordance with a preferred embodiment of the first aspect of the invention the method comprises determining the expression of (i) at least two nucleic acid molecules of the nucleotide sequences of SEQ ID NOs 6 to 10 or the nucleic acid molecules derived thereof as defined in the first aspect of the invention, (ii) at least two proteins of the amino acid sequence of any one of SEQ ID NOs 1 to 5 or the proteins or peptides derived thereof as defined in the first aspect of the invention, and/or (iii) at least one nucleic acid molecule of the nucleotide sequences of SEQ ID NOs 6 to 10 or the nucleic acid molecules derived thereof as defined claim 1 and at least one protein or peptide of the amino acid sequence of any one of SEQ ID NOs 1 to 5 or the proteins or peptides derived thereof as defined in the first aspect of the invention, is determined in step (A).

Tumors may not only express one of HLA-L, HLA-H, HLA-J, HLA-Y and HLA-V but also two or all three thereof in order to escape the immune system of the tumor patient. For this reason it is advantageous to determine with increasing preference the expression of at least two, at least, at least three, at least four and of all five of HLA-L, HLA-H, HLA-J, HLA-Y and HLA-V at the nucleic acid level, the protein level or any mixture thereof.

Measuring more than one of HLA-L, HLA-H, HLA-J, HLA-Y and HLA-V and optionally in additional one of more of the HLA genes, protein or peptides described herein is also since it allows compiling an anti-HLA treatment regimen which is optimized for the patient to be treated. With respect to the diagnostic measuring more than one of these HLAs allows determining an HLA expression profile, for example, in a selected tumor lesion.

In accordance with another preferred embodiment of the first aspect of the invention the method furthermore comprises determining in step (A) the expression of at least one of the HLA class Ib genes HLA-E, HLA-F, and HLA-G and/or at least one protein or peptide produced from said MHC class Ib genes.

In accordance with a further preferred embodiment of the first aspect of the invention, the method furthermore comprises determining in step (A) the expression of at least one of the HLA class I genes HLA-A, HLA-B, and HLA-C and/or at least one protein or peptide produced from said MHC class I genes.

In accordance with a yet further preferred embodiment of the first aspect of the invention, the method furthermore comprises determining in step (A) the expression of at least one of the HLA class II genes HLA-DPA1, HLA-DPB1, HLA-DQA1, HLA-DQB1, HLA-DRA, and HLA-DRB1 and/or at least one protein or peptide produced from said MHC class II genes.

The human leukocyte antigen (HLA) system or complex is a gene complex encoding the major histocompatibility complex (MHC) proteins in humans. These cell-surface proteins are responsible for the regulation of the immune system in humans. The HLA gene complex resides on a 3 Mbp stretch within chromosome 6p21. HLA genes are highly polymorphic, which means that they have many different alleles, allowing them to fine-tune the adaptive immune system. The proteins encoded by certain genes are also known as antigens, as a result of their historic discovery as factors in organ transplants.

The HLA system has been divided into three classes I to III. The two major classes are MHC classes I and II.

Humans have three main or classical MHC class I genes, known as HLA-A, HLA-B, and HLA-C. The main MHC class I genes are referred to in the art as class I or class Ia. The proteins produced from these genes are present on the surface of almost all cells. On the cell surface, these proteins are bound to protein fragments (peptides) that have been exported from within the cell. MHC class I proteins display these peptides to the immune system. Immune escape strategies aimed to avoid T-cell recognition, including the loss of tumor MHC class Ia expression, are commonly found in malignant cells (Garrido, Curr Opin Immunol. 2016 April; 39: 44-51). Hence, while tumor cells upregulate the expression of MHC class Iw in order to escape the immune system the expression of MHC class la is reduced by tumor cell. The therapy described herein may thus comprise compounds increasing the expression of HLA-A, HLA-B, and HLA-C. In the simplest form such compound can be HLA-A, HLA-B, and/or HLA-C or a vector or plasmid expressing HLA-A, HLA-B, and/or HLA-C. Since MHC class la is downregulated and MHC class Iw is upregulated by tumor cells in order to escape the immune system, the detection of each at least one member may also increase the selectivity of the method of the invention. This also because MHC class Ia is expressed by normal cells while MHC class Iw is to the best knowledge of the inventors not. Therefore, the boarders between normal and malignant tissue may be determined more selectively.

Humans furthermore have three minor or non-classical MHC class I genes, known as HLA-E, HLA-F, and HLA-G. The minor MHC class I genes are referred to in the art as class Ib. The HLA class Ib genes, HLA-E, HLA-F, and HLA-G, were discovered long after the classical HLA class Ia genes. Although results from a range of studies support the functional roles for the HLA class Ib molecules in adult life, especially HLA-G and HLA-F have most intensively been, and were also primarily, studied in relation to reproduction and pregnancy (Persson et al. (2017), Immunogenetics, DOI 10.1007/s00251-017-0988-4). The expression of HLA class Ib proteins at the feto-maternal interface in the placenta seems to be important for the maternal acceptance of the semi-allogenic fetus. In contrast to the functions of HLA class Ia, HLA class Ib possesses immune-modulatory and tolerogenic functions. HLA-F can be, for example, HLA-F1, -F2 or -F3. Similarly, HLA-G can be any one of HLA-G1, -G2, -G3, -G4, -G5, -G6, and -G7. In more detail, the primary transcript of HLA-G (8 Exons; NCBI Gene Bank NM_002127.5, version of Sep. 16, 2019) can be spliced into 7 alternative mRNAs that encode membrane-bound (HLA-G1, -G2, -G3, -G4) and soluble (HLA-G5, -G6, -G7) protein isoforms (Carosella et al., 2008, Trends Immunol.; 29(3):125-32). HLA-G1 is the full-length HLA-G molecule, HLA-G2 lacks exon 4, HLA-G3 lacks exons 4 and 5, and HLA-G4 lacks exon 5. HLA-G1 to -G4 are membrane-bound molecules due to the presence of the transmembrane and cytoplasmic tail encoded by exons 6 and 7. HLA-G5 is similar to HLA-G1 but retains intron 5, HLA-G6 lacks exon 4 but retains intron 5, and HLA-G7 lacks exon 4 but retains intron 3. HLA-G5 and -G6 are soluble forms due to the presence of intron 5, which contains a premature stop codon to prevent the translation of the transmembrane and cytoplasmic tail. HLA-G7 is soluble due to the presence of intron 3, which contains a premature stop codon. Also HLA-F is alternatively spliced. The three isoforms F1, F2 and F3 are all membrane-bound isoforms. No isoforms of HLA-E are reported and HLA-E is membrane-bound. The amino acids sequences of HLA-E, HLA.-F1, F2, F3 and HLA-G1, G2, G3, G4, G5, G6 and G7 are shown in SEQ ID NOs 13-23, respectively, and the nucleotide sequences encoding these amino acids sequences in SEQ ID NOs 24-34, respectively. The MHC class Ib proteins and peptides also comprise open conformer forms thereof as discussed herein above in connection with HLA-L. For instance, open conformers of HLA-F are known from the prior art; Garcia-Beltran (2016); Nat Immunol. 2016 September; 17(9):1067-1074. For instance, apart from the classical HLA conformation and complexes with other heavy HLA chains, there is a stable open conformation (OC) of HLA-F characterized by the absence of β2-microglobulin and peptides bound in the peptide binding groove (Sim et al. (2017) Immunity 2017, 46, 972-974). Some HLA genes, such as HLA-F form complexes with heavy chains of other HLA class I molecules (Goodridge et al (2010) J. Immunol. 2010, 184, 6199-6208). Also these open conformers and complexes are encompassed by the HLA class Ib genes that may be used in accordance with the invention. Just as in the case of MHC class Iw tumors upregulate the expression of MHC class Ib in order to escape the immune system (Würfel et. al. (2019), Int. J. Mol. Sci. 2019, 20, 1830; doi:10.3390/ijms20081830). The therapy described herein may thus comprise inhibitors of HLA-E, F and/or G. Preferred examples of types the class Iw inhibitors (e.g. antibodies, siRNA, small, antibody mimetics, molecules etc.) are described herein below and these preferred types apply mutatis mutandis to Ib inhibitors.

There are six main MHC class II genes in humans: HLA-DPA1, HLA-DPB1, HLA-DQA1, HLA-DQB1, HLA-DRA, and HLA-DRB1. MHC class II genes provide instructions for making proteins that are present almost exclusively on the surface of certain immune system cells. Like MHC class I proteins, these proteins display peptides to the immune system. MHC class II genes provide instructions for making proteins that are present almost exclusively on the surface of certain immune system cells. Like MHC class I proteins, these proteins display peptides to the immune system. The biological consequences of MHC-II expression by tumor cells are reviewed in Axelrod et al. (2019), Clin Cancer Res, DOI: 10.1158/1078-0432. Accumulating evidence demonstrates that tumor-specific MHC-II associates with favorable outcomes in patients with cancer, including those treated with immunotherapies, and with tumor rejection in murine models. For instance, the expression of MHC-II molecules on tumor cells can predict response to immune checkpoint blockade.

Human leukocyte antigen (HLA) molecules are mandatory for the immune recognition and subsequent killing of neoplastic cells by the immune system, as tumor antigens must be presented in an HLA-restricted manner to be recognized by T-cell receptors. Impaired HLA-Ia expression prevents the activation of cytotoxic immune mechanisms, whereas impaired HLA-II expression affects the antigen-presenting capability of antigen presenting cells. Aberrant HLA-Ib expression by tumor cells favors immune escape by inhibiting the activities of virtually all immune cells (Rodriguez (2017), Immunogenetics (2017), Oncology Letters, https://doi.org/10.3892/01.2017.6784, pages: 4415-4427 and EP 2 561 890 B1 and Würfel et al. (2019), Int. J. Mol. Sci. 2019, 20, 1830; doi:10.3390/ijms20081830).

In more detail, altered HLA-I(a) expression on the tumor cell surface is an early and frequent event that promotes carcinogenesis, as HLA-I(a) is critical for the immune recognition of tumor cells and signaling between tumor and immune cells. Several studies reported total or partial loss of classical HLA-I(a) molecule expression in different human tumors, with at least 50% of multiple HLA allele loss caused by loss of heterozygosity (LOH) events. Another HLA-mediated strategy used by tumor cells to avoid recognition by various immune effectors is the aberrant expression of minor or non-classical HLA-Ib molecule which function as inhibitor ligands for immune-competent cells, allowing tumor immune escape.

For the above reasons, it is advantageous to further assess in the method of the first aspect of the invention in addition the expression of one or more HLA class Ia, HLA class Ib and/or HLA class II genes or protein or peptides. The result of this expression analysis provides further information on how the tumor escapes the immune system of the subject and therefore further information which can be considered in order to provide the tumor patient with tailored medicament to combat the tumor or a tailored diagnostic agent to detect the tumor sites in vivo.

For instance, in case the expression of HLA class Ib is detected it is also preferred to incorporate into the medicament a compound being capable of inhibiting the detected HLA class Ib at the nucleic acid or protein level, just as described herein above in connection with the new HLA class Iw. Similarly, in case the expression of HLA class Ib is detected it is also preferred to incorporate into the diagnostic agent a compound being capable of detecting in vivo the sites and/or amounts of HLA class Ib at the nucleic acid or protein level, just as described herein above in connection with the new HLA class Iw. In this respect the preferred features and embodiments as described herein above and below in connection with the Iw class apply mutatis mutandis to the class Ib.

In accordance with another preferred embodiment of the first aspect of the invention, the method furthermore comprises determining in step (A) the expression of at least one growth factor and/or at least one tumor marker and/or at least one protein being expressed during early pregnancy and in carcinoembryonic regression.

A growth factor is a naturally occurring substance capable of stimulating cellular growth, proliferation, healing, and/or cellular differentiation. The role of growth factors in tumorigenesis and tumor progression is reviewed, for example, in Witsch et al. (2011), Physiology (Bethesda). 2010 April; 25(2): 85-101. Tumors aberrantly express growth factors with the effect that the tumor cells can grow in an uncontrolled manner. In case the expression of a growth factor is detected it is also preferred to incorporate into the medicament a compound being capable of inhibiting the detected growth factor at the nucleic acid or protein level, just as described herein above in connection with the new HLA class Iw. Similarly, in case the expression of a growth factor is detected it is also preferred to incorporate into the diagnostic agent a compound being capable of detecting in vivo the sites and/or amounts of the growth factor at the nucleic acid or protein level, just as described herein above in connection with the new HLA class Iw. In this respect the preferred features and embodiments as described herein above and below in connection with the Iw class apply mutatis mutandis to the growth factor.

A tumor marker is a biomarker found in blood, urine, or body tissues that can be elevated by the presence of one or more types of cancer. There are many different tumor markers, each indicative of a particular disease process, and they are used in oncology to help detect the presence of cancer. The tumor marker herein can be a nucleic acid molecule, protein, conjugated protein, or peptide. Hence, the detection of a tumor marker may aid in diagnosing a tumor. Accordingly, in case the expression of a tumor marker is detected it is also preferred to incorporate into the diagnostic agent a compound being capable of detecting in vivo the sites and/or amounts of the tumor marker at the nucleic acid or protein level, just as described herein above in connection with the new HLA class Iw. In this respect the preferred features and embodiments as described herein above and below in connection with the Iw class apply mutatis mutandis to the tumor marker.

Proteins being expressed during early pregnancy and in carcinoembryonic regression (e.g. the carcinoembryonic antigen (CEA) gene family) are normally not highly expressed after birth. The normal function of these proteins is organizing tissue architecture and regulating different signal transductions. Their aberrant expression leads to the development of human malignancies. In particular, CEA and CEACAM6 are up-regulated in many types of human cancers. Their aberrant expression is found in human malignancies. In case the expression of such a protein is detected it is also preferred to incorporate into the medicament a compound being capable of inhibiting the detected protein at the nucleic acid or protein level, just as described herein above in connection with the new HLA class Iw. Similarly, in case the expression of such a protein is detected it is also preferred to incorporate into the diagnostic agent a compound being capable of detecting in vivo the sites and/or amounts of the protein at the nucleic acid or protein level, just as described herein above in connection with the new HLA class Iw. In this respect the preferred features and embodiments as described herein above and below in connection with the Iw class apply mutatis mutandis to such protein.

In accordance with a more preferred embodiment of the first aspect of the invention, the at least one growth factor is selected from the group consisting of epidermal growth factor (EGF), fibroblast growth factor (FGF), basic fibroblast growth factor (bFGF), growth differentiation factor-9 (GDF9), hepatocyte growth factor (HGF), hepatoma-derived growth factor (HDGF), keratinocyte growth factor (KGF), nerve growth factor (NGF), placental growth factor (PGF), platelet-derived growth factor (PDGF), stromal cell-derived factor 1 (SDF1), transforming growth factor, and vascular endothelial growth factor.

The above list of growth factors is preferred since for these growth factors it is known that their expression drives tumor proliferation.

In accordance with another more preferred embodiment of the first aspect of the invention, the at least one tumor marker is selected from the group consisting of somatostatin receptors, TSH (thyrotropin) receptors, tyrosin receptors, and PSMA (prostate-specific membrane antigen).

The above list of tumor markers is preferred since these markers are currently used for the diagnosis of certain tumors.

In accordance with another preferred embodiment of the first aspect of the invention, (i) the medicament is or comprises a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, or a transcription activator-like (TAL) effector (TALE) nuclease capable of inhibiting the expression of the at least one nucleic acid molecule, and/or (ii) the medicament is or comprises a small molecule, an antibody, a protein drug, or an aptamer capable of inhibiting the at least one protein or peptide, wherein the protein drug is preferably an antibody mimetic, and wherein the antibody mimetic is preferably selected from affibodies, adnectins, anticalins, DARPins, avimers, nanofitins, affilins, Kunitz domain peptides and Fynomers®, and/or (iii) the diagnostic agent is or comprises a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease capable of binding to the at least one expressed nucleic acid molecule, and/or (iv) the diagnostic agent is or comprises a small molecule, an antibody, a protein drug, or an aptamer capable of binding to the at least one expressed protein or peptide, wherein the protein drug is preferably an antibody mimetic, and wherein the antibody mimetic is preferably selected from affibodies, adnectins, anticalins, DARPins, avimers, nanofitins, affilins, Kunitz domain peptides and Fynomers®.

The “small molecule” as used herein is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O. For all above-described organic molecules the molecular weight is preferably in the range of 200 Da to 1500 Da and more preferably in the range of 300 Da to 1000 Da.

Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 Da, or less than about 1000 Da such as less than about 500 Da, and even more preferably less than about Da amu. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

The term “antibody” as used in accordance with the present invention comprises, for example, polyclonal or monoclonal antibodies. Furthermore, also derivatives or fragments thereof, which still retain the binding specificity to the target, e.g. the HLA-J, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the protein in accordance with the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847). The use of such bispecific antibodies allows focusing the anti-tumor action of the bispecific antibodies to the tumor cells.

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope, for example, of HLA-J. Surface plasmon resonance as employed in the BlAcore system can be used to increase the efficiency of phage antibodies.

As used herein, the term “antibody mimetics” refers to compounds which, like antibodies, can specifically bind antigens, such the HLA-J protein, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and probodies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, e.g. against HLA-J, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, e.g. for HLA-J, can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. HLA-J, by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, e.g. against HLA-J, is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity, e.g. against HLA-J, can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the protein of the fourth aspect of the invention. The two other epitopes may also be epitopes of the protein of the fourth aspect of the invention or may be epitopes of one or two different antigens. The trispecific binding molecule is preferably a TriTac. A TriTac is a T-cell engager for solid tumors which comprised of three binding domains being designed to have an extended serum half-life and be about one-third the size of a monoclonal antibody.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop (SEQ ID NO: 35) in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

As used herein, the term “probody” refers to a protease-activatable prodrug, e.g. to a protease-activatable antibody prodrug. A probody, for example, consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects. It is furthermore possible to confine the binding and/or inhibitory activity of the small molecule, antibody or antibody mimetic and aptamer to certain tissues or cell-types, in particular diseased tissues or cell-types by probodies. In such a probody the small molecule, antibody or antibody mimetic or aptamer is also bound to a masking peptide which limits or prevents binding to the protein of the invention and which masking peptide can be cleaved by a protease. Proteases are enzymes that digest proteins into smaller pieces by cleaving specific amino acid sequences known as substrates. In normal healthy tissue, protease activity is tightly controlled. In cancer cells, protease activity is upregulated. In healthy tissue or cells, where protease activity is regulated and minimal, the target-binding region of the probody remains masked and is thus unable to bind. On the other hand, in diseased tissue or cells, where protease activity is upregulated, the target-binding region of the probody gets unmasked and is thus able to bind and/or inhibit.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor (for example, of HLA-J) after introduction into the respective cells.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

CRISPR/Cas9, as well as CRISPR-Cpf1, technologies are applicable in nearly all cells/model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific gene, here, for example, the HLA-J gene. Similarly, catalytically inactive, “dead” Cpf1 nuclease (CRISPR from Prevotella and Francisella-1) can be fused to synthetic transcriptional repressors or activators to downregulate endogenous promoters, e.g. the promoter which controls, for example, HLA-J expression. Alternatively, the DNA-binding domain of zinc finger nucleases (ZFNs) or transcription activator-like effector nucleases (TALENs) can be designed to specifically recognize the target (e.g. HLA-J) gene or its promoter region or its 5-UTR thereby inhibiting the expression of the target.

Inhibitors provided as inhibiting nucleic acid molecules that target the gene of interest or a regulatory molecule involved in its expression are also envisaged herein. Such molecules, which reduce or abolish the expression of the target gene or a regulatory molecule include, without being limiting, meganucleases, zinc finger nucleases and transcription activator-like (TAL) effector (TALE) nucleases. Such methods are described in Silva et al., Curr Gene Ther. 2011; 11(1):11-27; Miller et al., Nature biotechnology. 2011; 29(2):143-148, and Klug, Annual review of biochemistry. 2010; 79:213-231.

In accordance with a more preferred embodiment of the first aspect of the invention, the small molecule, antibody, protein drug, or aptamer being or comprised in the medicament is fused to a cytotoxic agent, wherein the cytotoxic agent is preferably a therapeutic radioisotope, more preferably ¹⁷⁷Lu, ⁹⁰Y, ⁶⁷Cu and ²²⁵Ac, and/or small molecule, antibody, protein drug, or aptamer being the or comprised in the diagnostic agent is fused to an imaging agent, wherein the imaging agent is preferably a therapeutic radioisotope, more preferably ⁶⁷Ga, ⁴⁴⁵Sc, ¹¹¹In, ^{99m}Tc, ⁵⁷Co, ¹³¹I.

According to this preferred embodiment the small molecule, antibody, protein drug, or aptamer is to be generated in the format of a conjugate. Cleavable and non-cleavable linkers to design conjugates are known in the art.

In this case the small molecule, antibody, protein drug, or aptamer in itself may not have an inhibitory effect but the inhibitory effect is only conferred by the conjugation partner. Similarly, the small molecule, antibody, protein drug, or aptamer in itself may not be capable to detect tumor sites in vivo but said detection is only enabled by the conjugation partner.

In these cases the small molecule, antibody, protein drug, or aptamer confers the site-specificity binding of the medicament or diagnostic agent to the protein or peptide according to the invention.

In the case of a medicament the cytotoxic agent is capable to kill cells producing and/or binding to the protein according to the invention. Hence, by combining the targeting capabilities of molecules binding to the protein according to the invention with the cell-killing ability of the cytotoxic agent, the conjugates become inhibitors that allow for discrimination between healthy and diseased tissue and cells. Similarly, in the case of a diagnostic agent the diagnostic agent is capable to detect (e.g. make visible) cells producing and/or binding to the protein according to the invention. Hence, by combining the targeting capabilities of molecules binding to the protein according to the invention with the cell-killing ability of the diagnostic agent, the conjugates become diagnostic agents that allow for the detection of tumor sites in vivo.

Therapeutic radioisotopes deliver radiation directly to tumor cells in an amount that kills the cancer cells. Examples of such isotopes are ¹⁷⁷Lu, ⁹⁰Y, ⁶⁷Cu and ²²⁵Ac. On the other hand, therapeutic radioisotopes deliver radiation directly to tumor cells only in an amount that allows detecting the radiation via radiodiagnostics. Examples of such isotopes are ⁶⁷Ga, ⁴⁴Sc, ¹¹¹In, ^{99m}Tc, ⁵⁷Co, ¹³¹I.

The present invention relates in a second aspect to a medicament produced by the method of the first aspect of the invention for use in the treatment or prevention of a tumor in a subject.

The subject to be treated is preferably the same subject from whom the sample has been obtained being used in the method of the first aspect of the invention. As a result the subject receives a tumor treatment or prevention which is tailored to the tumor in the subject.

The present invention relates in a third aspect to a diagnostic agent produced by the method of the first aspect of the invention for use in the in vivo detection of tumor sites in a subject.

The subject to be diagnosed is preferably the same subject from whom the sample has been obtained being used in the method of the first aspect of the invention. As a result the subject receives a tumor diagnosis which is tailored to the tumor in the subject.

In accordance with a preferred embodiment of the third aspect of the invention, the detection comprises scanning the entire body of the subject, wherein the scanning preferably employs a total-body positron emission tomography (PET) scanner.

Whole body scanners, such as a total-body positron emission tomography (PET) scanner, produce pictures, preferably 3D-pictures of the entire human body.

The PET scanner is particularly advantageous because of the scanner's high efficiency. It is able to produce images in as little as a second using a standard radiation dose, much faster than with conventional devices. Moreover, to help reduce radiation exposure, the dose can be reduced at the expense of just a few extra seconds of the scanner's time. The scanner can assess how different tissues and organs react to different stimuli. The spread of inflammation, the impact of different disorders, and the mobility of cancer tumors can also be easier assessed using this scanning technology. The PET scanner is preferably the Explorer Total Body Scan™.

In accordance with a preferred embodiment of the third aspect of the invention, the detection comprises measuring the radiation dose uptake of the radioisotope into the tumor sites in a subject.

In accordance with this embodiment a diagnostic radioisotope, more preferably ⁶⁷Ga, ⁴⁴Sc, ¹¹¹In, ^{99m}Tc, ⁵⁷Co, ¹³¹I is used in the detection. The radioisotope is preferably part of a conjugate as described herein above.

Means and methods for measuring radiation dose uptake of a radioisotope into the tumor sites in a subject are known in the art, for example, from Eberle Huguette et al. (2014) World J Nucl Med.; 13(1): 50-55 or Francis et al. (2015) Journal of Radiation Research and Applied Sciences, 8(2):182-189. For example, a Siemens e.cam SPECT system can be used for imaging and the quantification of the uptake based on the images may be performed by a software, e.g. the Image J software.

In accordance with a more preferred embodiment of the third aspect of the invention, based on the measured radiation dose uptake a therapeutically effective amount of a medicament is to be determined, wherein the medicament is preferably produced by the method of the first aspect of the invention.

Quantification of radionuclide uptakes in tumors is important and recommended in assessing patient's response to therapy, doses to critical organs and in diagnosing tumors (Francis et al. (2015) Journal of Radiation Research and Applied Sciences, 8(2):182-189). For instance, a low uptake a diagnostic radioisotope indicates that a higher do of the therapeutic radioisotope is needed and vice versa.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

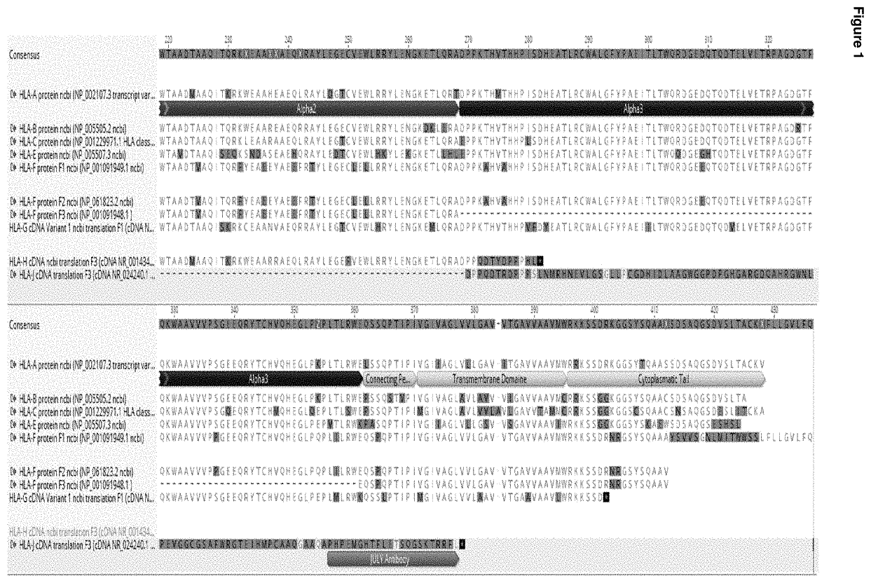

FIG. 1: Summary of the protein sequences of HLA-A, HLA-B, HLA-C, HLA-E, HLA-F isoforms, HLA-G, HLA-H and HLA-J in the region of the alpha 2 and 3 domains, the transmembrane domains and the corresponding connecting peptide with the cytoplasmatic region. The consensus sequence is highlighted in grey above the aligned sequences. Differences in the HLA peptide sequences are also highlighted in grey. The predicted alpha 3 differs from the other HLA genes and is highlighted in grey. The peptide sequence for the generation of the unique HLA-J antibody is depicted as brown arrow, named “JULY Antibody”.

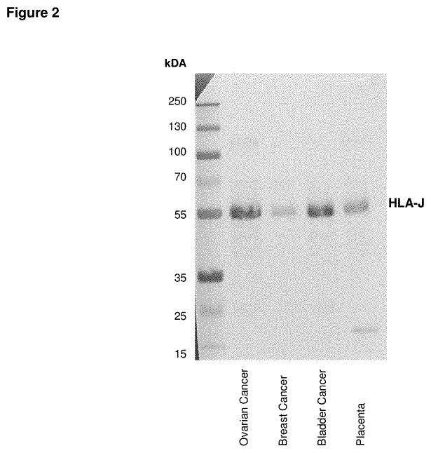

FIG. 2: Evidence of HLA-J protein expression by western blot analysis in ovarian cancer, breast cancer and bladder cancer tissue from patients as well as placental tissue. The examples illustrate the invention.

The examples illustrate the invention.

### EXAMPLE 1

### Imaging of Cancer Cells by Using Radionuclide-Labelled Anti-HLA-Antibodies

The example describes the generation of a personalized anti-tumor therapy with the aid of radionuclide labelled antibodies also denoted as theranostics. It also describes an in vivo method for detecting and treating tumor and metastases in patients via positron emission tomography (PET) and computed tomography (CT).

In a first step, the individual and unique HLA expression pattern (adult and/or embryonic and/or former “pseudogenes”) of the tumor and metastases are visualized with anti-HLA antibodies labelled to a diagnostical radionuclide. After the determination of the HLA expression pattern and evaluating cancer cell distribution and tumor burden, a therapeutic, personalized anti-HLA antibody mixture labelled to a therapeutical radionuclide is applied. Therapy response and success can be monitored with the anti-HLA antibodies labelled to the diagnostical radionuclide, which have been applied in the first step.

In order to minimize radiation exposure and maximize the therapeutic effect of the applied radio-labelled anti-HLA-antibody, uptake kinetics, it is advantageous to determine the HLA status previous to therapy.

PET/CT imaging is performed with the whole-body scanner “Explorer Total Body Scanner™” (United Imaging, Shanghai) in order to determine HLA expression pattern. Compared to other PET/CT-Scanner this system has a 40-times higher sensitivity with a 30 second total measuring time. It detects lesions that express an HLA having a size of 2.8 mm with a 6-times higher imaging resolution. The whole-body scanner also lowers the radiation burden to patients and minimizes side effects.

The antibodies, which are applied in order to detect HLA class Ib and Iw genes, which are solely expressed on tumor cells e.g. anti-HLA-G antibodies (named “LILLY1” and “LILLY2”) and anti-HLA-J antibody (named “JULY”). These antibodies are generated against peptide sequences which are unique for each HLA class Ib and Iw gene in order to minimize cross reactivity to other HLA genes (BioGenes, Berlin, Germany). The HLA-J antibody has been generated against the c-terminal end of the unique alpha 3 and transmembrane domain of HLA-J (FIG. 1). The peptide sequence includes 22 amino acids spanning the alpha3 domain, the connecting peptide and the n-terminal end of the transmembrane domain.

### EXAMPLE 2

### Detection of HLA-J Protein Expression

In order to proof the existence of the protein of HLA-J western blot analysis has been performed in ovarian cancer, breast cancer and bladder cancer tissue from patients and placenta (n=1). 20 μg of protein tissue lysates were separated in a 10% SDS-PAGE gel under denatured conditions and transferred wet to a nitrocellulose membrane. After incubation with the specific anti-HLA-J antibody “JULY”, purple precipitates have been observed after incubation with an anti-rabbit antibody coupled with HPR, followed by the application of TMB substrate. Western blot analysis revealed the existence of a HLA-J protein with the observed size of approximately 55 kDa (FIG. 2). The calculated protein size of HLA-J is around 26.7 kDa. Regarding the ovarian cancer tissue sample, further bands can be detected at around 100 kDa. These findings might indicate that HLA-J mainly exists in a dimer and tetramer conformations, caused by cysteine residues which can create disulfide bonds.

### EXAMPLE 3

### Labelling of Anti-HLA-J JULY-mAb and Anti-HLA-G LILLY-mAb With ⁶⁸Gallium for Imaging

In order to fully evaluate cancer cell distribution, the HLA-J antibody JULY and the anti-HLA-G LILLY were labelled to the diagnostic radionuclide, Gallium-68.

Gallium-68, an amphoteric element, was derived from a Ge-68/Ga-68 generator system from the parent nuclide germanium-68 with a long half-life of 288 days according to the current state of knowledge (Zhernosekov et al., J Nucl Med 2007 October; 48(10):1741-8). In order to obtain radio-chemically pure gallium-68, cation exchange post-processed to Ge-68/Ga-68 generator has been performed enabling the collection of pure gallium-68 within 10 minutes (Mueller et al., Recent Results Cancer Res. 2013; 194:77-87). Since gallium-68 itself has a half-life of approximately 68 minutes fast labelling has to be carried out. Gallium-68 was labelled to JULY and LILLY with the chelator DOTA (1,4,7,10-tetraazacyclododecane-N,N′,N″,N″′-tetraacetic acid) based on the cassette-based synthesis system click chemistry (EZAG, Berlin, Germany). This system provides an automated, fast and GMP compliant production method for the generation of radiopharmaceuticals. The quality, sterility, endotoxin testing as well as chemical and radio-chemical purity were tested according to the monographs from the European Pharmacopeia, V.8.0 (European Pharmacopoeia (Ph. Eur.) Vol 8 (2013-2016) European Directorate of Quality of Medicines). Biodistribution, binding affinity and dosimetry were checked in vivo in cell lines as well on fresh-frozen tissue slides from patients.

The requirements for Lutetium-177 labelled JULY and LILLY therapies are similar to Lutetium-177 PSMA treatment (Baum et al., Nuklearmediziner 2015; 38(02): 145-152). Kidney protection was performed according to the bad berka protocol (Schuchardt et al., Recent Results Cancer Res. 2013; 194:519-36).

### EXAMPLE 4

### Labelling of Anti-HLA-J JULY-mAb and Anti-HLA-G LILLY With ¹⁷⁷Lutetium for Therapy

Lutetium-177 is a lower energy beta emitting radionuclide with a mean penetration range of 650 μm in soft tissue and a half-life of 6.72 days. The small range of this beta emitting radionuclide, but 50× greater ranger to alpha emitting radionuclides makes lutetium-177 an optimal therapeutical radionuclide. The emission of low energy gamma doses enables its imaging and distribution analysis via PET/CT. It is generated by the indirect production route over ytterbium-176 according to the patent DE102011051868A1. Labelling of the antibodies JULY and LILLY was performed as described in Repetto-Llamazares et al, PLoS One. 2014; 9(7).

The quality, sterility, endotoxin testing as well as chemical and radio-chemical purity were tested according to the monographs from the European Pharmacopeia, V.8.0 (European Pharmacopoeia (Ph. Eur.) Vol 8 (2013-2016) European Directorate of Quality of Medicines). Biodistribution, binding affinity and dosimetry were checked in cell lines as well on fresh-frozen tissue slides from patients.

### EXAMPLE 5

### Biodistribution of Anti-HLA-J JULY-mAb and Anti-HLA-G Lilly-mab Radiolabelled ⁶⁸Gallium In Vivo After Systemic Application Via Intravenous Injection Into the Tail Vein and Instillation Into the Bladder in BBN Induced Bladder Cancer Carcinogenesis Animal Models

Experimental Animal Set Up

Bladder cancer was induced with the bladder specific carcinogen N-butyl-N-(4-hydroxybutyl) nitrosamine (BBN) in C57BU6/c mice (Charles River Laboratories International, Inc, Wilmington, Mass.) according to George et al (Transl Oncol. 2013 June; 6(3): 244-255). In brief, animals were divided into two groups (n=27-30/group). Group 1 served as the control, which received only tap water, whereas Group 2 was treated with BBN. The BBN (TCI America, Portland, Oreg.) carcinogen was supplied ad libitum at 0.05% in drinking water to mice from 8 to 20 weeks of age. Water consumption was recorded to determine BBN intake and compared between groups. Body weights were measured at multiple time points between 8 and 32 weeks of age. Animals were monitored for tumor progression and survival and were killed after 32 weeks to obtain bladder and organ weights

Biodistribution with Anti-HLA-J JULY-mAb and Anti-HLA-G Lilly-Mab Radiolabelled ⁶⁸Gallium:

Biodistribution was assessed in animals which had successfully developed bladder cancer as well as in tumor-free mice. Animals were intravesically injected with 6.66 MBq of Gallium-68 labelled anti-HLA-J JULY-mAb or Gallium-68 labelled anti-HLA-G Lilly-mab in 100 μL of PBS. Forty-five and 90 min after injection, mice were sacrificed and organs were prepared for measurement of Gallium-68 labelled anti-HLA-J JULY-mAb or Gallium-68 labelled anti-HLA-G Lilly-mAb accumulation in a γ-counter. Uptake was expressed as percentage of injected activity per gram of tissue. Bladders were isolated and split in half with one portion processed for histology and immunohistochemistry and the other flash frozen for RNA isolation.

A second set up was performed in order to determine the systemic biodistribution by applying the Gallium-68 labelled anti-HLA-J JULY-mAb or Gallium-68 labelled anti-HLA-G Lilly-mAb systemic via intravenous injection into the tail vein. Forty-five and 90 min after injection, mice were sacrificed and organs were prepared for measurement of Gallium-68 labelled anti-HLA-J JULY-mAb or Gallium-68 labelled anti-HLA-G Lilly-mAb accumulation in a γ-counter. Uptake was expressed as percentage of injected activity per gram of tissue. Bladders were isolated and split in half with one portion processed for histology and immunohistochemistry and the other flash frozen for RNA isolation.

Biodistribution was monitored with a PET-CT scanner.

Radioimmunotherapy with Anti-HLA-J JULY-mAb and Anti-HLA-G Lilly-mAb Radiolabelled ¹⁷⁷Lutetium:

Radioimmunotherapy was performed with tumor-bearing mice and divided into 9 groups consisting of 10 animals each. These groups received 0.925 MBq of Lutetium-177 anti-HLA-J JULY-mAb or Lutetium-177 anti-HLA-G LILLY-mAb in 100 μL of PBS intravesically at 1 h, 7 d, or 14 d after BBN induction or 0.37 MBq of Lutetium-177 anti-HLA-J JULY-mAb or 68-Gallium anti-HLA-G LILLY-mAb in 100 μL of PBS at 1 h or 7 d after BBN induction, or 40 pg mitomycin C in 40 μL of 0.9% NaCl at 1 h or 7 d, after BBN induction, or 2 μg of unlabeled Lutetium-177 anti-HLA-J JULY-mAb or Lutetium-177 anti-HLA-G LILLY-mAb at 1 h after BBN induction. The control group received PBS intravesically at 1 h after BBN induction. During therapy, mice were anesthetized (90 min).

A second set up was performed by applying Lutetium-177 anti-HLA-J JULY-mAb or 68-Gallium anti-HLA-G LILLY-mAb systemic via intravenous injection into the tail vein. Tumor bearing mice were divided into 9 groups consisting of 10 animals each. These groups received 0.925 MBq of Lutetium-177 anti-HLA-J JULY-mAb or Lutetium-177 anti-HLA-G LILLY-mAb in 100 μL of PBS intravenous at 1 h, 7 d, or 14 d after BBN induction or 0.37 MBq of Lutetium-177 anti-HLA-J JULY-mAb or 68-Gallium anti-HLA-G LILLY-mAb in 100 μL of PBS at 1 h or 7 d after BBN induction, or 40 μg mitomycin C in 40 μL of 0.9% NaCl at 1 h or 7 d, after BBN induction, or 2 μg of unlabeled Lutetium-177 anti-HLA-J JULY-mAb or Lutetium-177 anti-HLA-G LILLY-mAb at 1 h after BBN induction. The control group received PBS intravesically at 1 h after tumor cell inoculation. During therapy, mice were anesthetized (90 min).

Radioimmunotherapy was monitored with a PET-CT scanner.

Histopathologic Evaluation and Tissue Microarray Preparation

To assess bladder histopathology, urinary bladders were first excised, cut in half longitudinally, and fixed in 10% buffered formalin. Formalin-fixed bladders were then paraffin embedded, sectioned, and stained with hematoxylin and eosin following standard protocols. Stained slides were histopathologically graded by an expert pathologist (S.S.S.), and bladders were categorized into normal or cancerous, invasive or muscleinvasive, bladders. These were then reviewed to mark the area for tumor for the construction of tissue microarrays. Tissue microarrays were made using 0.6-mm cylindrical cores punched out from the original paraffin blocks using a manual tissue arrayer (Beecher Instruments, Silver Spring, Md.). Triplicate cores from individual blocks were made to enhance the representative reproducibility. Thus, a total of 540 cores representing 180 female mice were used to generate five master blocks. Five-micrometer sections were cut from these blocks and placed on charged slides (Fisher Scientific, Houston, Tex.) and stained appropriately. Briefly, these slides were deparaffinized, rehydrated, and pretreated by either microwave or proteinase K for antigen retrieval. Immunohistochemical staining was then performed using corresponding antibodies. The staining procedure was based on an indirect biotin-avidin system with a universal biotinylated Ig secondary antibody, DAB substrate, and hematoxylin counterstain. A negative control slide was obtained after either omitting the primary antibody or incubating with an irrelevant antibody (mouse monoclonal Ig).

Tumor Cell Proliferation by Ki-67 Staining

Using the tissue microarrays generated above, sections were also stained for Ki-67 antigen assessed by immunohistochemistry using a monoclonal MIB-1 antibody (clone MIB-1, mouse IgG1, 1:100 from Dako North America Inc, Carpinteria, Calif.) that was incubated for 25 minutes in a TechMate 500 Plus (Dako North America Inc) and visualized with DAB. Images were captured using the Vectra scanner using the CRI multispectral camera with a ×20 magnification objective (Caliper, Hopkinton, Mass.) for the entire tissue section. Image analysis was done using InForm 1.2 software. InForm was trained to count the Ki-67-positive cells in representative fields for each tissue section. From the images, areas of tissue other than urothelia were masked using Image-Pro Plus software (Media Cybernetics Inc, Bethesda, Md.). The percentage of positively stained cells was calculated using images for the entire section of tissue.

Apoptosis Assays

Cell death was detected in situ by enzymatic labeling of DNA strand breaks using terminal deoxynucleotidyl transferase-mediated dUTP nick end labeling (TUNEL) assays as described previously. For negative controls, the terminal deoxynucleotidyl transferase was substituted by deionized water, while sections that were pretreated with 1.0 g/ml DNase I (DN 25; Sigma-Aldrich, St Louis, Mo.) were used for the positive controls. Images were captured using Vectra scanner as described above, and percentage of TUNEL-positive cells was determined using InForm 1.2 and Image-Pro Plus software.

HLA Immunohistochemistry

For immunohistochemical staining of HLA-J and HLA-G, rabbit polyclonal antibodies against HLA-J and HLA-G (BioGenes, Berlin, Germany) were used. Assessment of HLA-J and HLA-G expression was performed by a pathologist (S.S.S.) blinded to the tissue treatment using a modified version of Allred scoring. A composite score, ranging from 0 to 9, was obtained by multiplying the percentage grade by the intensity. HLA-J and -G expression scores were grouped as negative (0), low (<6), and high (≥6).

Analyses of HLA mRNA Expression

Bladder specimens obtained from male mice were powdered and homogenized with QlAshredder columns (Qiagen, Hilden, Germany) and total RNA was extracted with the RNeasy Mini Kit (Qiagen, Hilden, Germany) according to the manufacturer's recommendations. RNA was quantitated by quantitative real-time polymerase chain reaction (qPCR) analyses using TaqMan primer and probes. All extracts were tested for sufficient high-quality RNA content by quantification with real time PCR (RT-qPCR) of the constitutively expressed gene Calmodulin 2 gene (CALM2) which is known as a stable reference/housekeeper gene. For a detailed analysis of gene expression by RT-qPCR methods, primers flanking the region of interest and a fluorescently labeled probe hybridizing in-between were utilized. RNA-specific primer/probe sequences were used to enable RNA-specific measurements by locating primer/probe sequences across exon/exon boundaries. In case multiple isoforms of the same gene existed, primers were selected to amplify all relevant or selected splice variants as appropriate. All primer pairs were checked for specificity by conventional PCR reactions. Specific primers have been generated against HLA-H, J, L and G (Table 1).

| Gen | For_Primer | Probe | Rev-Primer |
|---|---|---|---|
| HLA-G- | GGCCGGAGTATTGGGAAGA | CAAGGCCCACGCACAGACTGA | GCAGGGTCTGCAGGTTCATT |
| Ex3 | | CA | |
| HLA-G | CTGCGGCTCAGATCTCCAA | CGCAAGTGTGAGGCGGCCAAT | CAGGTAGGCTCTCCTTTGTT |
| Ex4 | | | CAG |
| HLA-G | CACCACCCTGTCTTTGACT | ACCCTGAGGTGCTGGGCCCTG | AGTATGATCTCCGCAGGGTA |
| Ex5 | ATGAG | | GAAG |
| HLA-G | CATCCCCATCATGGGTATC | TGCTGGCCTGGTTGTCCTTGC | CCGCAGCTCCAGTGACTACA |
| Ex6 | G | A | |
| HLA-G | GACCCTCTTCCTCATGCTG | CATTCCTTCCCCAATCACCTT | CATCCCAGCCCCTTTTCTG |
| Ex8 | AAC | TCCTGTT | |
| HLA-G | TTCATCGCCATGGGCTACG | CGACACGCAGTTCGTGCGGTT | ATCCTCGGACACGCCGAGT |
| Ex3-5 | | C | |
| HLA-G | CCGAACCCTCTTCCTGCTG | CGAGACCTGGGCGGGCTCCC | GCGCTGAAATACCTCATGGA |
| Ex2/3 | C | | |
| HLA-H | GAGAGAACCTGCGGATCGC | AGCGAGGGCGGTTCTCACACC | CCACGTCGCAGCCATACAT |
| Ex2/3 | | ATG | |
| HLA-H | GAGAGAACCTGCGGATCGC | ACCAGAGCGAGGGCGGTTCTC | CGGGCCGGGACATGGT |
| | | ACAC | |
| KRT5 | CGCCACTTACCGCAAGCT | TGGAGGGCGAGGAATGCAGAC | ACAGAGATGTTGACTGGTCC |
| | | TCA | AACTC |
| KRT20 | GCGACTACAGTGCATATTA | TTGAAGAGCTGCGAAGTCAGA | CACACCGAGCATTTTGCAGT |
| | CAGACAA | TTAAGGATGCT | T |
| CALM2 | GAGCGAGCTGAGTGGTTGT | TCGCGTCTCGGAAACCGGTAG | AGTCAGTTGGTCAGCCATGC |
| | G | C | T |
| HLA-L | CCTGCTCCGCTATTACAAC | CGAGGCCGGTATGAACAGTTC | CGTTCAGGGCGATGTAATCC |
| Ex2/3 | CA | GCCTA | |
| HLA-L | GCTGTGGTTGCTGCTGCG | AGAAAAGCTCAGGCAGCAATT | CATAGTCCTCTTTACAAGTA |
| Ex5/6 | | GTGCTCAG | TCATGAGATG |
| HLA-L | TCCTCTTCTGCTCAGCTCT | CTCTCCCTTCCCTGAGTTGTA | GCTTTATAGATCCATGAGTT |
| Ex7 | CCTA | GTAATCCTAGCACT | TGCATTA |
| HLA-J | CAAGGGGCTGCCCAAGC | CATCCTGAGATGGGTCACACA | CCTCCTAGTCTTGGAACCTT |
| Ex4/5 | | TTTCTGGAA | GAGAAGT |

The above primers and probes correspond to SEQ ID NOs 36 to 83. For instance the forward primer, the probe and the reverse primer of HLA-G Exon 3 are SEQ ID NOs 36, 37, and 38, respectively.

Results

Treatment with the bladder specific carcinogen BBN resulted in 70% tumor growth. Forty-five and 90 min after intravesical instillation of Gallium-68 labelled anti-HLA-J JULY-mAb or Gallium-68 labelled anti-HLA-G Lilly-mAb (6.66 MBq in 100 μL), the uptake of the radioimmunoconjugate in the different organs was analyzed via quantification of ⁶⁸Gallium activity. As presumed, locoregional intravesical application of Gallium-68 labelled anti-HLA-J JULY-mAb or Gallium-68 labelled anti-HLA-G Lilly-mAb ensured excellent retention of the therapeutic compound in the bladder with negligible systemic activity. These data suggest low systemic toxicity, as confirmed after the sacrifice of animals surviving more than 300 d without any signs of disease.

To monitor therapeutic response and efficacy after intravesical Lutetium-177 anti-HLA-J JULY-mAb and Lutetium-177 anti-HLA-G LILLY-mAb treatment, PET-CT images of tumors were recorded at different time points before and after therapy. After the application of Lutetium-177 anti-HLA-J JULY-mAb and Lutetium-177 anti-HLA-G LILLY-mAb treatment 14 d after BBN induction, both complete eradication and decrease of tumor burden could be observed. Additionally, light emissions from tumors of selected mice were quantified over ROIs before and after therapy using Simple PCI software. Light emissions of intravesical tumors of mice treated with Lutetium-177 anti-HLA-J JULY-mAb or Lutetium-177 anti-HLA-G LILLY-mAb treatment, (0.925 MBq) at 7 d after BBN induction, indicate complete or partial remission of intravesical tumors.

Mice that were treated with PBS or unlabeled anti-HLA-J JULY-mAb and anti-HLA-G LILLY-mAb treatment 1 h after tumor cell instillation reached a median survival of 41 and 89 d, respectively. Groups that underwent Lutetium-177 anti-HLA-J JULY-mAb and Lutetium-177 anti-HLA-G LILLY-mAb treatment therapy with 0.37 or 0.925 MBq 1 h after BBN induction both showed a significantly longer median survival of more than 300 d (P<0.001) and did not develop any tumor. A disease-free survival was observed in 90% of the animals.

### EXAMPLE 6

### Biodistribution of Anti-HLA-J JULY-mAb and Anti-HLA-G Lilly-mAb Radiolabelled ⁶⁸Gallium and Therapy With These Antibodies Radiolabelled Lutetium-177 After Instillation Into the Bladder of an Advanced, Muscle-Invasive Bladder Cancer Patient Not Responding to Neoadjuvant Platium Based Chemotherapy

Biodistribution was performed with Gallium-68 labelled anti-HLA-J JULY-mAb and Gallium-68 labelled anti-HLA-G Lilly-mAb antibodies in patients suffering from advanced, muscle-invasive bladder cancer which did not respond to neoadjuvant platium based chemotherapy. Instillation was applied according to the EAU urology guidelines (Roupret et al., Eur Urol. 2018 January; 73(1):111-122). Biodistribution was monitored with a whole-body PET-CT scanner. Molecular imaging with PET-CT revealed that ⁶⁸Gallium labelled anti-HLA-J JULY-mAb and anti-HLA-G Lilly-mAb majorly targeting muscle-invasive bladder cancer with a simultaneously very low uptake to the surrounding healthy bladder tissue. This data indicates low systemic toxicity of anti-HLA-J JULY-mAb and anti-HLA-G LILLY-mAb, resulting in an effective anti-tumor therapy.

For radioimmunotherapy patients received lutetium-177 labelled anti-HLA-J JULY-mAb and anti-HLA-G LILLY-mAb. Instillation was applied according to the EAU urology guidelines (Roupret et al., Eur Urol. 2018 January; 73(1):111-122). The advantage of an instillation therapy with lutetium-177 labelled anti-HLA-J JULY-mAb and anti-HLA-G LILLY-mAb compared to its systemic application via the venous system is the systemic lower radiotoxical burden as well as preservation of the kidney function. All patients had a histological proven muscle-invasive bladder cancer. Instillation therapy response was monitored with an whole body PET-CT scanner. Reconstruction was performed using the iterative reconstruction algorithm implemented by the manufacturer including attenuation and scatter correction based on the low dose CT. For quantitative analysis, the dynamic list mode data were reconstructed as 6 images of 300 s. Mean standardized uptake value (SUV) were measured in fixed size volumes of interest (VOIs) in the bladder as well as in all organs.

HLA-J and HLA-G positive bladder cancer were detected. No adverse side effects were observed. The acquired images obtained with the Lutetium-177 labelled anti-HLA-J JULY-mAb and anti-HLA-G LILLY-mAb were similar to the previously obtained images using Gallium-68 labelled anti-HLA-J JULY-mAb and Gallium-68 labelled anti-HLA-G Lilly-mAb antibodies. PET-CT Images were taken at different time points in order to evaluate the therapeutic response. It could be observed that patients showed response to anti-HLA-J and anti-HLA-G radioimmunotherapy by a decrease in tumor size as well as pathological complete response.

### EXAMPLE 7

### Biodistribution of Anti-HLA-J JULY-mAb and Anti-HLA-G Lilly-mAb Radiolabelled ⁶⁸Gallium and Therapy With These Antibodies Radiolabelled Lutetium-177 Intravenously Injected Into an Advanced, Muscle-Invasive Bladder Cancer Patient Not Responding to Neoadjuvant Platium Based Chemotherapy

Biodistribution was performed with Gallium-68 labelled anti-HLA-J JULY-mAb and Gallium-68 labelled anti-HLA-G Lilly-mAb antibodies in patients suffering from advanced, muscle-invasive bladder cancer which did not respond to neoadjuvant platium based chemotherapy. Intravenous injection was applied according to the EAU urology guidelines (Roupret et al., Eur Urol. 2018 January; 73(1):111-122).

Biodistribution was monitored with a whole-body PET-CT scanner. Molecular imaging with PET-CT revealed that ⁶⁸Gallium labelled anti-HLA-J JULY-mAb and anti-HLA-G Lilly-mAb majorly targeting muscle-invasive bladder cancer with a simultaneously very low uptake to other organs. This data indicates low systemic toxicity of anti-HLA-J JULY-mAb and anti-HLA-G LILLY-mAb, resulting in an effective anti-tumor therapy.

For radioimmunotherapy patients received lutetium-177 labelled anti-HLA-J JULY-mAb and anti-HLA-G LILLY-mAb. Intravenous injection was applied according to the EAU urology guidelines (Roupret et al., Eur Urol. 2018 January; 73(1):111-122). All patients had a histological proven muscle-invasive bladder cancer. Radioimmunotherapy response was monitored with a whole body PET-CT scanner. Reconstruction was performed using the iterative reconstruction algorithm implemented by the manufacturer including attenuation and scatter correction based on the low dose CT. For quantitative analysis, the dynamic list mode data were reconstructed as 6 images of 300 s. Mean standardized uptake value (SUV) were measured in fixed size volumes of interest (VOIs) in the bladder as well as in all organs.

HLA-J and HLA-G positive bladder cancer were detected as well as different metastatic sides with a simultaneously very low uptake to other organs. No adverse side effects were observed. The acquired images obtained with the Lutetium-177 labelled anti-HLA-J JULY-mAb and anti-HLA-G LILLY-mAb were similar to the previously obtained images using Gallium-68 labelled anti-HLA-J JULY-mAb and Gallium-68 labelled anti-HLA-G Lilly-mAb antibodies. PET-CT Images were taken at different time points in order to evaluate the therapeutic response. It could be observed that patients showed response to anti-HLA-J and anti-HLA-G radioimmunotherapy by a decrease in tumor size as well as pathological complete response. In addition, metastases did also decrease in size and number. Some patients showed total pathological complete response to the applied radioimmunotherapy by a total absence of any metastases or tumor.

### EXAMPLE 8

### Biodistribution of Anti-HLA-J JULY-mAb and Anti-HLA-G Lilly-mAb Radiolabelled ⁶⁸Gallium and Therapy With These Antibodies Radiolabelled Lutetium-177 After Instillation Into the Bladder of a Non Muscle Invasive Bladder Cancer Patient Refractory to BCG Instillation

Biodistribution was performed with Gallium-68 labelled anti-HLA-J JULY-mAb and Gallium-68 labelled anti-HLA-G Lilly-mAb antibodies in patients suffering from advanced, non-muscle-invasive bladder cancer which did not respond to neoadjuvant platium based chemotherapy. Instillation was applied according to the EAU urology guidelines (Roupret et al., Eur Urol. 2018 January; 73(1):111-122). Biodistribution was monitored with a whole-body PET-CT scanner. Molecular imaging with PET-CT revealed that ⁶⁸Gallium labelled anti-HLA-J JULY-mAb and anti-HLA-G Lilly-mAb majorly targeting muscle-invasive bladder cancer with a simultaneously very low uptake to the surrounding healthy bladder tissue. This data indicates low systemic toxicity of anti-HLA-J JULY-mAb and anti-HLA-G LILLY-mAb, resulting in an effective anti-tumor therapy.

For radioimmunetherapy patients received lutetium-177 labelled anti-HLA-J JULY-mAb and anti-HLA-G LILLY-mAb. Instillation was applied according to the EAU urology guidelines (Roupret et al., Eur Urol. 2018 January; 73(1):111-122). The advantage of an instillation therapy with lutetium-177 labelled anti-HLA-J JULY-mAb and anti-HLA-G LILLY-mAb compared to its systemic application via the venous system is the systemic lower radiotoxical burden as well as preservation of the kidney function. All patients had a histological proven non-muscle-invasive bladder cancer. Instillation therapy response was monitored by whole body PET-CT scanner. Reconstruction was performed using the iterative reconstruction algorithm implemented by the manufacturer including attenuation and scatter correction based on the low dose CT. For quantitative analysis, the dynamic list mode data were reconstructed as 6 images of 300 s. Mean standardized uptake value (SUV) were measured in fixed size volumes of interest (VOls) in the bladder as well as in all organs.

HLA-J and HLA-G positive bladder cancer were detected, but not in any other organ. No adverse side effects were observed. The acquired images obtained with the Lutetium-177 labelled anti-HLA-J JULY-mAb and anti-HLA-G LILLY-mAb were similar to the previously obtained images using Gallium-68 labelled anti-HLA-J JULY-mAb and Gallium-68 labelled anti-HLA-G Lilly-mAb antibodies. PET-CT Images were taken at different time points in order to evaluate the therapeutic response. It could be observed that patients showed response to anti-HLA-J and anti-HLA-G radioimmunotherapy by a decrease in tumor size as well as pathological complete response.

### EXAMPLE 9

### Biodistribution of Anti-HLA-J JULY-mAb and Anti-HLA-G Lilly-mAb Radiolabelled ⁶⁸Gallium and Therapy With These Antibodies Radiolabelled Lutetium-177 Intravenously Injected to a Non Muscle Invasive Bladder Cancer Patient Refractory to BCG Instillation

Biodistribution was performed with Gallium-68 labelled anti-HLA-J JULY-mAb and Gallium-68 labelled anti-HLA-G Lilly-mAb antibodies in patients suffering from advanced, non-muscle-invasive bladder cancer which did not respond to BCG treatment. Intravenous injection was applied according to the EAU urology guidelines (Roupret et al., Eur Urol. 2018 January; 73(1):111-122). Biodistribution was monitored with a whole-body PET-CT scanner. Molecular imaging with PET-CT revealed that ⁶⁸Gallium labelled anti-HLA-J JULY-mAb and anti-HLA-G Lilly-mab majorly targeting non-muscle-invasive bladder cancer with a simultaneously very low uptake to other organs. This data indicates low systemic toxicity of anti-HLA-J JULY-mAb and anti-HLA-G LILLY-mAb, resulting in an effective anti-tumor therapy.

For radioimmunotherapy patients received lutetium-177 labelled anti-HLA-J JULY-mAb and anti-HLA-G LILLY-mAb. Intravenous injection was applied according to the EAU urology guidelines (Roupret et al., Eur Urol. 2018 January; 73(1):111-122). All patients had a histological proven non-muscle-invasive bladder cancer. Radioimmunotherapy response was monitored with a whole-body PET-CT scanner. Reconstruction was performed using the iterative reconstruction algorithm implemented by the manufacturer including attenuation and scatter correction based on the low dose CT. For quantitative analysis, the dynamic list mode data were reconstructed as 6 images of 300 s. Mean standardized uptake value (SUV) were measured in fixed size volumes of interest (VOIs) in the bladder as well as in all organs.

HLA-J and HLA-G positive bladder cancer were detected as well as different metastatic sides with a simultaneously very low uptake to other organs. No adverse side effects were observed. The acquired images obtained with the Lutetium-177 labelled anti-HLA-J JULY-mab and anti-HLA-G LILLY-mab were similar to the previously obtained images using Gallium-68 labelled anti-HLA-J JULY-mAb and Gallium-68 labelled anti-HLA-G Lilly-mab antibodies. PET-CT Images were taken at different time points in order to evaluate the therapeutic response. It could be observed that patients showed response to anti-HLA-J and anti-HLA-G radioimmunotherapy by a decrease in tumor size as well as pathological complete response. In addition, metastases did also decrease in size and number. Some patients showed total pathological complete response to the applied radioimmunotherapy by a total absence of any metastases or tumor.

1. A method for producing a medicament for the treatment or prevention of a tumor in a subject or a diagnostic agent for the detection of a tumor in a subject comprising
(A) determining the expression of at least one nucleic acid molecule and/or at least one protein or peptide in a sample obtained from said subject,
wherein the at least one nucleic acid molecule is selected from nucleic acid molecules
(a) encoding a polypeptide comprising or consisting of the amino acid sequence of any one of SEQ ID NOs 1 to 5,
(b) comprising or consisting of the nucleotide sequence of any one of SEQ ID NOs 6 to 10,
(c) encoding a polypeptide which is at least 85% identical, preferably at least 90% identical, and most preferred at least 95% identical to the amino acid sequence of (a),
(d) consisting of a nucleotide sequence which is at least 95% identical, preferably at least 96% identical, and most preferred at least 98% identical to the nucleotide sequence of (b),
(e) consisting of a nucleotide sequence which is degenerate with respect to the nucleic acid molecule of (d),
(f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and
(g) corresponding to the nucleic acid molecule of any one of (a) to (f), wherein T is replaced by U, and
wherein the at least one protein or peptide is selected from proteins or peptides being encoded by the nucleic acid molecule of any one of (a) to (g); and
(B) producing a medicament capable of inhibiting the expression of the at least nucleic acid molecule and/or the at least one protein or peptide in the subject, if the at least one nucleic acid molecule and/or at least one protein or peptide is expressed in (A), and/or
(B′) producing a diagnostic agent capable of detecting in vivo the sites of expression of the at least nucleic acid molecule and/or the at least one protein or peptide in the subject, if the at least one nucleic acid molecule and/or at least one protein or peptide is expressed in (A).

2. The method of claim 1, wherein the expression of
(i) at least two nucleic acid molecules of the nucleotide sequences of SEQ ID NOs 6 to 10 or the nucleic acid molecules derived thereof as defined claim 1,
(ii) at least two proteins of the amino acid sequence of any one of SEQ ID NOs 1 to 5 or the proteins or peptides derived thereof as defined claim 1, and/or
(iii) at least one nucleic acid molecule of the nucleotide sequences of SEQ ID NOs 6 to 10 or the nucleic acid molecules derived thereof as defined claim 1 and at least one protein or peptide of the amino acid sequence of any one of SEQ ID NOs 1 to 5 or the proteins or peptides derived thereof as defined claim 1,
is determined in step (A).

3. The method of claim 1 or 2, furthermore determining in step (A) the expression of at least one of the HLA class Ib genes HLA-E, HLA-F, and HLA-G and/or at least one protein or peptide produced from said MHC class Ib genes.

4. The method of any one of claims 1 to 3, furthermore determining in step (A) the expression of at least one of the HLA class I genes HLA-A, HLA-B, and HLA-C and/or at least one protein or peptide produced from said MHC class I genes.

5. The method of any one of claims 1 to 4, furthermore determining in step (A) the expression of at least one of the HLA class II genes HLA-DPA1, HLA-DPB1, HLA-DQA1, HLA-DQB1, HLA-DRA, and HLA-DRB1 and/or at least one protein or peptide produced from said MHC class II genes.

6. The method of any one of claims 1 to 5, furthermore determining in step (A) the expression of at least one growth factor and/or at least one tumor marker and/or at least one protein being expressed during early pregnancy and in carcinoembryonic regression.

7. The method of claim 6, wherein the at least one growth factor is selected from the group consisting of epidermal growth factor (EGF), fibroblast growth factor (FGF), basic fibroblast growth factor (bFGF), growth differentiation factor-9 (GDF9), hepatocyte growth factor (HGF), hepatoma-derived growth factor (HDGF), keratinocyte growth factor (KGF), nerve growth factor (NGF), placental growth factor (PGF), platelet-derived growth factor (PDGF), stromal cell-derived factor 1 (SDF1), transforming growth factor, and vascular endothelial growth factor.

8. The method claim 6, wherein the at least one tumor marker is selected from the group consisting of somatostatin receptors, TSH receptors, tyrosin receptors, and PSMA.

9. The method of any one of claims 1 to 8, wherein
(i) the medicament is or comprises a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, or a transcription activator-like (TAL) effector (TALE) nuclease capable of inhibiting the expression of the at least one nucleic acid molecule, and/or
(ii) the medicament is or comprises a small molecule, an antibody, a protein drug, or an aptamer capable of inhibiting the at least one protein or peptide,
wherein the protein drug is preferably an antibody mimetic, and
wherein the antibody mimetic is preferably selected from affibodies, adnectins, anticalins, DARPins, avimers, nanofitins, affilins, Kunitz domain peptides and Fynomers®, and/or
(iii) the diagnostic agent is or comprises a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease capable of binding to the at least one expressed nucleic acid molecule, and/or
(iv) the diagnostic agent is or comprises a small molecule, an antibody, a protein drug, or an aptamer capable of binding to the at least one expressed protein or peptide,
wherein the protein drug is preferably an antibody mimetic, and
wherein the antibody mimetic is preferably selected from affibodies, adnectins, anticalins, DARPins, avimers, nanofitins, affilins, Kunitz domain peptides and Fynomers®.

10. The method of claim 9, wherein the
small molecule, antibody, protein drug, or aptamer being or comprised in the medicament is fused to a cytotoxic agent, wherein the cytotoxic agent is preferably a therapeutic radioisotope, more preferably ¹⁷⁷Lu, ⁹⁰Y, ⁶⁷Cu and ²²⁵Ac, and/or
small molecule, antibody, protein drug, or aptamer being the or comprised in the diagnostic agent is fused to an imaging agent, wherein the imaging agent is preferably a therapeutic radioisotope, more preferably ⁶⁷Ga, 44Sc, ¹¹¹In, ^{99m}Tc, ⁵⁷Co, ¹³¹I.

11. A medicament produced by the method of any one claims 1 to 10 for use in the treatment or prevention of a tumor in a subject.

12. A diagnostic agent produced by the method of any one claims 1 to 10 for use in the in vivo detection of tumor sites in a subject.

13. The diagnostic agent for use of claim 12, wherein the detection comprises scanning the entire body of the subject, wherein the scanning preferably employs a total-body positron emission tomography (PET) scanner.

14. The diagnostic agent for use of claim 12 or 13, wherein the detection comprises measuring the radiation dose uptake of the radioisotope into the tumor sites in a subject.

15. The diagnostic agent for use of claim 14, wherein based on the measured radiation dose uptake a therapeutically effective amount of a medicament is to be determined, wherein the medicament is preferably produced by the method of any one claims 1 to 10.


SEQUENCE LISTING (83 sequences)

SEQ ID NO: 1 - protein, 221 aa, Homo sapiens
  HLA-H soluble
MVLMAPRTLLLLLSGALALTLTQTWARSHSMRYFYTTMSRPGRGEPRFISVGYVDDTQFVRFDSDDASPREEPRAPWMEREGPEYWDRNTQICKAQAQTERENLRIALRYYNQSEGGSHTMQVMYGCDVGPDGRFLRGYEQHAYDGKDYIALNEDLRSWTAADMAAQITKRKWEAARRAEQLRAYLEGEFVEWLRRYLENGKETLQRADPPQDTYDPPPHL

SEQ ID NO: 2 - protein, 219 aa, Homo sapiens
  HLA-J soluble
MAPRTLLLLLSGTLALAETWAGSHSMRYFSTAVSWPGRGEPSFIAVGYVDDTQFVRVDSDAVSLRMKTRARWVEQEGPEYWDLQTLGAKAQAQTDRVNLRTLLRYYNQSEADPPQDTRDPPPSLNMRHNEVLGSGLLPCGDHIDLAAGWGGPDPGHGARGDQAHRGWNLPEVGGCGSAFWRGTEIHMPCAAQGAAQAPHPEMGHTFLETSRGSKTRRFL

SEQ ID NO: 3 - protein, 246 aa, Homo sapiens
  HLA-L soluble
MGVMAPRTLLLLLLGALALTETWAATPVRGWSGGRRGWSRRGWSIGTRRHGTPRATRRFTETCGPCSAITTRARPVTVRLRWQGLHRPERGPALLDRREHSGSDLPAQVGSGQILRAGQGLPEGKCMEWLRRHLENGKETLQHADPPKAHVTQHPISDHEATLRCWALGLYPAEITLTWQQDGEDQTQDTELVETRPAGDGTFQKWVAVVVPSGEEQRYMCHVQHEGLPEPLTLRWEPSSQPTIPI

SEQ ID NO: 4 - protein, 111 aa, Homo sapiens
  HLA-V soluble
MAPRTLLLLLSGALVLTQTWAGFHSLRYFHTTMSRPGRADPRFLSVGDVDDTQCVRLDSDATSPRMEPRAPWMEQEGPEYWEEETGTAKAKAQFYRVNLRTLSGYYNQSEA

SEQ ID NO: 5 - protein, 206 aa, Homo sapiens
  HLA-Y soluble
MAVVAPRTLLLLLSGALALTQTWAGSHSMRYFSTSVSRPGSGEPRFIAVGYVDDTQFVRFDSDAASQRMEPRAPWMEQEEPEYWDRQTQISKTNAQIDLESLRIALRYYNQSEAGSHTIQRMSGCDVGSDGRFLRGYRQDAYDGKDYIALNEDLRSWTAADMAAQITQRKWEAARQAEQLRAYLEGECMEWLRRYLENGKETLQRT

SEQ ID NO: 6 - DNA, 1704 bp, Homo sapiens
  HLA-H soluble
agtttctcttcttctcacaacctgcgacgggtccttcttccttgatactcacgaagcggacacagttctcattcccactaggtgtcgggtttctagagaagccaatcggtgccgccgcggtcccggttctaaagtccccacgcacccaccgggactcagattctccccagacgccgaggatggtgctcatggcgccccgaaccctcctcctgctgctctcaggggccctggccctgaccctgacccagacctgggcgcgctcccactccatgaggtatttctacaccaccatgtcccggcccggccgcggggagccccgcttcatctccgtcggctacgtggacgatacgcagttcgtgcggttcgacagcgacgacgcgagtccgagagaggagccgcgggcgccgtggatggagcgggaggggccagagtattgggaccggaacacacagatctgcaaggcccaggcacagactgaacgagagaacctgcggatcgcgctccgctactacaaccagagcgagggcggttctcacaccatgcaggtgatgtatggctgcgacgtggggcccgacgggcgcttcctccgcgggtatgaacagcacgcctacgacggcaaggattacatcgccctgaacgaggacctgcgctcctggaccgcggcggacatggcagctcagatcaccaagcgcaagtgggaggcggcccgtcgggcggagcagctgagagcctacctggagggcgagttcgtggagtggctccgcagatacctggagaacgggaaggagacgctgcagcgcgcggaccccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccgagcccctcaccctgagatgggagccatcttcccagcccaacgtccccatcgtgggcatcgttgctggcctggttctacttgtagctgtggtcactggagctgtggtcgctgctgtaatgtggaggaagaagagctcagatagaaaaggagggagctactctcaggctgcaacagcaacagtgcccagggctctgatgtgtctctcacggcttgaaagtgtgagacagctgccttgtgtgggactgagaggcaagagttgttcctgccttccctttgtgacttgaagaaccctgactttctttctacaaaggcacctgaatgtgtctgtgttcctgtaggcataatgtgtggaggaggggagaccaacccaccctcatgtccaccatgaccctcttccccacgctgatctgtgttccctccccaatcatctttcctgttccagagaggaggggctgagatgtctccatctttttctcaactttatgtgcactgagctgtaacttcttacttccctcttaaaattagaatctgagtaaacatttactttttcaaattcttgccatgagaggttgatgacttaattaaaggagaagattcctaaaatttgagagacaaaataaatggaacacatgagaaccttccag

SEQ ID NO: 7 - DNA, 1552 bp, Homo sapiens
  HLA-J soluble
ctatactatctcatgcacccaggcacaacttttccagatttaaagaaaaagaaaaaagaaataaaagaaaaaaacctctgtctctacacctccattcccagggagagctccctctctggcaccaagctccctggggtgagttttctttttgaagagtccaggggaacagcctgcgacgggtccttcttcctggacactcacgacgcggacccagttctcactcccactgagtgtcgggttttagggaagccaatcagcgtcgcgcggccccggttctaaagtccccacgcacccaccgggactcggagtctccccagacgccgacgatggggtcatggcgccccgaaccctcctcctgctgctctcggggaccctggccctggccgagacctgggcgggctcccactccatgaggtatttcagcaccgccgtttcctggccgggccgcggggagcccagcttcattgccgtgggctacgtggacgacacgcagttcgtgcgggtcgacagtgacgccgtgagtctgaggatgaagacgcgggcgcggtgggtggagcaggaggggccggagtattgggacctacagacactgggcgccaaggcccaggcacagactgaccgagtgaacctgcggaccctgctccgctactacaaccagagcgaggcggaccccccccaagacacacgtgacccacccccctctctgaacatgaggcataacgaggtcctgggttctgggcttctaccctgcggagatcacattgacctggcagcgggatggggaggaccagacccaggacatggagctcgtggagaccaggcccacaggggatggaaccttccagaagtgggcggttgtggtagtgccttctggagaggaacagagatacacatgccatgtgcagcacaaggggctgcccaagcccctcatcctgagatgggtcacacatttctggaaacttctcaaggttccaagactaggaggttcctctaggacctcatggccctgctaccttcctggcctctcacaggacgttttcttcccgcagatagaaaaggagggagctactctcaggctgcaagcagccaaagtgcccagggctctgatgtgtctctcacggcttgtaaagtgtgagacagctgccttgtgtgggactgagaggcaagatttgttcatgccttccctttgtgacttcaagaaccctgacttctctttctgcaaaggcatctgaatgtgtctgtgtccctataggcataatgtgaggtggtggggagaccagcccacacccgtgtccaccatgaccctgttccccacactgacctacattccttccccgatcacctttcctgttccagagaagtggtgctgggatgtctccatctctgtctcaacttcatggtgcactgagctgtaacttcttacttccctattaaaattagaatctgagtataaatttacttttttcaaattatttccatgacgggttgatgggttaattaaaggagaagattcctaaaatttgagagacaaaataaatggaagacatgagaa

SEQ ID NO: 8 - DNA, 898 bp, Homo sapiens
  HLA-L soluble
acgatcccggcactacagtcccggcgcaaccacccgcactcagattctccccaaacgccaaggatgggggtcatggctccccgaaccctcctcctgctgctcttgggggccctggccctgaccgagacctgggccgcgactccgtgagtccgaggatggagcggcgggcgccgtgggtggagcaggaggggctggagtattgggaccaggagacacggaacgccaagggccacgcgcagatttaccgagtgaacctgcggaccctgctccgctattacaaccagagcgaggccggtatgaacagttcgcctacgatggcaaggattacatcgccctgaacgaggacctgcactcctggaccgccgcgaacacagcggctcagatctcccagcacaagtgggaagcggacaaatactcagagcaggtcagggcctacctgagggcaagtgcatggagtggctccgcagacacctggagaacgggaaggagacgctgcagcacgcggatcccccaaaggcacatgtgacccagcaccccatctctgaccatgaggccaccctgaggtgctgggccctgggcctctaccctgcggagatcacactgacctggcagcaggatggggaggaccagacccaggacacggagcttgtggagaccaggcctgcaggggacggaaccttccagaagtgggtggctgtagtggtgccttccggagaggagcagagatacatgtgccatgtgcagcatgaggggctgccagagcccctcaccctgagatgggagccgtcttctcagcccaccatccccatcgtgggcatcgttgctggcctgtttctccttggagctgtggtcactggagctgtggttgctgctgcgatgtggaggaagaaaagctcag

SEQ ID NO: 9 - DNA, 1285 bp, Homo sapiens
  HLA-V soluble
gaaacattgagacagagcgcttggcacagaagtagcggggtcagggcgaagtcccagggcctcaggcgtggctctcaggatctcaggccccaaaggcggtgtatggattggggaggcccagcgctgggcattccccatctttgcagggtttctcttctccctctcccaacctgtgtcgggtccttcttcctgggtactcaccgggctgccccagttctcactcccattgagtgtcgggtttctagagaagccaatcaatgtagccgcggtcccggttctaaagttcccacgcacccaccgggactccgattcttcccagtcgccgaggatggtgtcatggcgccccgaaccctgcttctgctgctctcgggggccctggtcctgacccagacctgggcaggcttccactccttgaggtatttccacaccaccatgtcccggcccggccgcgcggatccccgcttcctctccgtgggcgacgtggacgacacgcagtgcgtgcggctcgacagcgacgccacgagtcccaggatggagccgcgggcgccgtggatggagcaggaggggccggaatattgggaagaggagacagggaccgccaaggccaaagcacagttttaccgagtgaacctgcggaccctgagcggctactacaaccagagtgaggcctaagtgcagcttcattccctccctgttcgtgtggcctggacttaatgactcacttctaactgatagagtaatgctgacataatagtttgtgattctgggtgtagaacataagactcactgaagtttctactttggttctttctttctctggaatcatgagccctgggggaagctggctgttgtgtcataaggaggcctgtggtccatgtgactaggaagtgagtcctcctgggaccagacaataagaagctaaagcctcttccaaaagccatgtgagagattcttgtgtcttgtgaatccccggccccatttgagccctcagatgattcagccctggaagacaactagactgcaacgttgtgagaggccctgagccagaagcattcagagaaacttctcctggattcctgaccatggataactgtgggagatgataaatatttgttgatttgagctgctaagttgtaggtgacttgttatgcagcagtagataactaatacagcttcacaagagaggatgaatcactgaactttttcatttgctctaaattcattataagatattaaacatgtcatttgcttttaatatttaataaaaatttccatggctatataa

SEQ ID NO: 10 - DNA, 1095 bp, Homo sapiens
  HLA-Y soluble
atggcggtcgtggcgccccgaaccctcctcctgctactctcgggggccctggccctgacccagacctgggcgggctcccactccatgaggtatttctccacatccgtgtcccggcccggcagtggagagccccgcttcatcgcagtgggctacgtggacgacacgcagttcgtgcggttcgacagcgacgccgcgagccagaggatggagccgcgggcgccgtggatggagcaggaggagccggagtattgggaccggcagacacagatctccaagaccaacgcacagattgacctagagagcctgcggatcgcgctccgctactacaaccagagcgaggccggttctcacaccatccagaggatgtctggctgcgacgtggggtcggacgggcgcttcctccgcgggtaccggcaggacgcctacgacggcaaggattacatcgccctgaacgaggacctgcgctcttggaccgcggcggacatggcggctcagatcacccagcgcaagtgggaggcggcccgtcaggcggagcagttgagagcctacctggagggcgagtgcatggagtggctccgcagatacctggagaacgggaaggagacgctgcagcgcacggacccccccccaagacacatatgacccaccaccccatctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctgcccaagcccctcaccctgagatgggagccgtcttcccagcccaccatccccatcgtgggcatccttgctggcctggttctctttggagctgtgatcgctggagctgtggtcgctgctgtgatgtggaggaggaagagctcagatagaaaaggagggagctactctcaggctgcaagcagtgacagtgcccagggctctgatgtgtctctcacagcttgtaaag

SEQ ID NO: 11 - protein, 341 aa, Homo sapiens
  HLA-L membrane-bound
MGVMAPRTLLLLLLGALALTETWAATPVRGWSGGRRGWSRRGWSIGTRRHGTPRATRRFTETCGPCSAITTRARPVTVRLRWQGLHRPERGPALLDRREHSGSDLPAQVGSGQILRAGQGLPEGKCMEWLRRHLENGKETLQHADPPKAHVTQHPISDHEATLRCWALGLYPAEITLTWQQDGEDQTQDTELVETRPAGDGTFQKWVAVVVPSGEEQRYMCHVQHEGLPEPLTLRWEPSSQPTIPIVGIVAGLFLLGAVVTGAVVAAAMWRKKSSGSNCAQYSDASHDTCKEDYACSCSGVCVLISFSPGCPSSLTAAGVIFPVINPTRWKAAPAHRSLWY

SEQ ID NO: 12 - DNA, 4622 bp, Homo sapiens
  HLA-L membrane-bound
acgatcccggcactacagtcccggcgcaaccacccgcactcagattctccccaaacgccaaggatgggggtcatggctccccgaaccctcctcctgctgctcttgggggccctggccctgaccgagacctgggccgcgactccgtgagtccgaggatggagcggcgggcgccgtgggtggagcaggaggggctggagtattgggaccaggagacacggaacgccaagggccacgcgcagatttaccgagtgaacctgcggaccctgctccgctattacaaccagagcgaggccggtatgaacagttcgcctacgatggcaaggattacatcgccctgaacgaggacctgcactcctggaccgccgcgaacacagcggctcagatctcccagcacaagtgggaagcggacaaatactcagagcaggtcagggcctacctgagggcaagtgcatggagtggctccgcagacacctggagaacgggaaggagacgctgcagcacgcggatcccccaaaggcacatgtgacccagcaccccatctctgaccatgaggccaccctgaggtgctgggccctgggcctctaccctgcggagatcacactgacctggcagcaggatggggaggaccagacccaggacacggagcttgtggagaccaggcctgcaggggacggaaccttccagaagtgggtggctgtagtggtgccttccggagaggagcagagatacatgtgccatgtgcagcatgaggggctgccagagcccctcaccctgagatgggagccgtcttctcagcccaccatccccatcgtgggcatcgttgctggcctgtttctccttggagctgtggtcactggagctgtggttgctgctgcgatgtggaggaagaaaagctcaggcagcaattgtgctcagtactctgatgcatctcatgatacttgtaaagaggactatgcctgttcctgttctggtgtctgcgttctgatctctttctcccctgggtgtccctcatctctgacagcagcaggagtcatttttcctgtcattaaccccacaaggtggaaggcagcccctgcacacagaagtctgtggtattaagagatgaattttcaagcccgtgcagcttttaccctatttccagggctctttcttggattgtattttctatcttttccccaacctttttaaaggaactagattctgaaattagcagagaagagggatgccacaagttctcatcttaggtaactttctagtggaactcctcttctgctcagctctcctacccactctcccttccctgagttgtagtaatcctagcactggctctaatgcaaactcatggatctataaagcaaagtctaacttagatttatatttgtttggaaattgggattcatagtcaaagattgttctttcctaagagggaaatataattgcatgctgcagtgtgcagagggttggtgtgaaggagggatgcagggaggaagggagggaggacacacaagcagcactgctgggaaaagcacaggcggcctggatgtcagtgtgaggggaccttgtgctgtcgttgctgcaaaaccgcatttggcctgaggctatgttaataaagatactgcctttagaataggaggtgctctacagtgatgattcattcagccgacatttgctgtctgccagacatatgacagaatgtttttgcatctggggaaagtcattgaagtaaaatcagaaaaatctctagccttgtggagcatgtgttccagtgggaagaggcagacggtacatacactctaatatatgcagagtaaatgaggaaagtgttagaaggtgataagtgctgtggaacaggtgatcagagtatgggttgtgggacagagaaggtagctattgtgccggggttgtcagcgtgggccttgttgggaaggtgacctttgatgaaatatttgaaggacataaaggaatttgtcatgagggtatctggaagaagttttttctagggagtaggaaccttcagtgtcagtgtaccagggcaggatcatgtctgtgtgttctgggaagaacacgggatcgggtatggctagagcagagagtcactgagataaggtcaggggtttggtcagatcatgtgggcatagggctcaagtatgtgggaaggattttgattttgaatgagatagttttaagcagaataaagacatgccacaacttctcttttaaaaggatcactgtagctgctctgctgagaacagaatccaaaggccggcgatgagcaaggcaggtgggaaaactgtaggaaatgagtgcagtatttcaggctggagatgtcggttacttcaactggggtgtgagcagtggaaatagtgggacgtgattggattcctactatttccaatcactttataccgcattttctaatggactaaatctggggtatgagaaagaagagtaaaggataccaaaaatgtcagactgtgactaaaaagagttgccatcagctgagaatgagaagactagcaggagcatatgagaggaggggacgtcgcaggcagtcactatgggagacgtgggatctgagatgccgctgagaaataccagtgaggtagtcgggttggcagttggacagatgaatctggagacatttaggagaaatagacttgggaggtgatgtcatataacagttatttaaagccttgagtctgaatgacgtctccaagggagtgattggctgtagaagagaacaggaacaaggactgaacactaggcctctgttgctaaaggatctgatcagacaacacacctagatcagactgcacagtcctgaccccacatctagaaggtacatagaccagggagttctagactttcctgtggacaggaatcacctggacatcaccttaagtctaagctgatctggaatcgagaatgagatttcctacttatataatgttgctgttggcgctgatgctgctggtcttcagatcccacttttggtagcaagaacacagaccaggattcctaggctatgcatcagcctcgcctgtgaggcttgttaataagcaattcctgcactccatgcgcaacattctgacacaggggcatctgtggagaggcctgagtattctacaacaagcccacagcaaacctggtgctcagccagatttgatatcactgagatcagtagttggagaatgcccaggatggggaggggtctcagacccacatttaagtgttgctttattctgggttttttatttatttatttatttatttttaaggaggatgtgtttctttaattataagacaggatgctgagagataaatgtcattttctctatcatggggtatagccagatggaagattgagaagtggctcacagctcagcagaatgaaaaaatatctgaatgctgctttctgaaactactctccagaatgatttcacactcactccttggagcaaacaatgacttgcaaatttttctaatttaaacataaaggagtgtacatattggtattagtattcattttattttggggaagggcactgtattagtccatagtccgttttcacactgccgataaagacatacccaacattgggaagaaaaagaggtttaattggacttacagttccatttggctggggaggcctcagaatcatggtgggaggcgaaaggcacttcttacatggtggtggcaagagaaaatgaggaagaagcaaatgccaaaacccctgataaacacattggatctcaggagacttattcattatcatgagaatagcatgggaaagactggcccccatgattcaattacctccccctgggtccctcccacaacatgtgggaattctgggagatacaattcaagttgagatttgggtggggacacagccaaaccacattggacacagaaccaggtttgaagctacacagccaggaacataatccacagccaccctaattcagatctctcataggaaccactgtccctgctcctgagcacagatgctactgcatatacctctgataccctgatggccgacactgggccctgtggcaaagactgctatcactgctgctcctgagaactgctccactactgctcctcagccatctttaccaaaatgcagtatttactgtcccagcctctctgtgtcatctcatcctgattagaagcccacatgtggttatctaaattgtgcagccaaagcctcttgcagtgtttaactgcaataatgttggggaaagtgaatttttctcctttgtagaaggaggtagtccctgccttctaataagactcttcaacataggaagagaattcagttgctggaggtagaggggtgagggatggaaaaagaatgacaaatttcaattcctagaatcatgttctgagactagaactttatctagtacattgcaggcacctgggtttggttgagtgtataataaatgacatagttcaacttattcccttgacagtttgttttggggtccagcttttgtctaccccagttttcacacacagatacgtggagaagcattgtgtgatggtaaaatgtttacttgaaagcctttttccctatctttgtctcttgctaggattaaaaacccgtatctgt

SEQ ID NO: 13 - protein, 357 aa, Homo sapiens
  HLA-E
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHS

SEQ ID NO: 14 - protein, 442 aa, Homo sapiens
  HLA-F1
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAAYSVVSGNLMITWWSSLFLLGVLFQGYLGCLRSHSVLGRRKVGDMWILFFLWLWTSFNTAFLALQSLRFGFGFRRGRSFLLRSWHHLMKRVQIKIFD

SEQ ID NO: 15 - protein, 346 aa, Homo sapiens
  HLA-F2
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 16 - protein, 254 aa, Homo sapiens
  HLA-F3
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRAEQSPQPTIPIVGIVAGLVVLGAVVTGAVVAAVMWRKKSSDRNRGSYSQAAV

SEQ ID NO: 17 - protein, 337 aa, Homo sapiens
  HLA-G1
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 18 - protein, 245 aa, Homo sapiens
  HLA-G2
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 19 - protein, 129 aa, Homo sapiens
  HLA-G3
MVVMAPRTLFLLLSGALTLTETWASHSMRYFSAAVSRPGRGEPRFIAMGYVDDTQFVRFDSDSACPRMEPRAPWVEQEGPEYWEEETRNKQSSLPTIPIMGIVAGLVVLAAVVTGAAVAAVLWRKKSSD

SEQ ID NO: 20 - protein, 245 aa, Homo sapiens
  HLA-G4
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRAKQSSLPTIPIMGIVAGLVVLAAVVTGAAVAAVLWRKKSSD

SEQ ID NO: 21 - protein, 318 aa, Homo sapiens
  HLA-G5
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 22 - protein, 226 aa, Homo sapiens
  HLA-G6
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 23 - protein, 115 aa, Homo sapiens
  HLA-G7
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASE

SEQ ID NO: 24 - DNA, 2548 bp, Homo sapiens
  HLA-E
ctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaagaacctggatctcaatttaatttttcatattcttgcaatgaaatggacttgaggaagctaagatcatagctagaaatacagataattccacagcacatctctagcaaatttagcctattcctattctctagcctattccttaccacctgtaatcttgaccatataccttggagttgaatattgttttcatactgctgtggtttgaatgttccctccaacactcatgttgagacttaatccctaatgtggcaatactgaaaggtggggcctttgagatgtgattggatcgtaaggctgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccagggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgaggccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaactttgaagtaaaataaaagctgtgtttgagcatca

SEQ ID NO: 25 - DNA, 1480 bp, Homo sapiens
  HLA-F1
atatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagcctactcagtggtcagcggaaacttgatgataacatggtggtcaagcttatttctcctgggggtgctcttccaaggatatttgggctgcctccggagtcacagtgtcttgggccgccggaaggtgggtgacatgtggatcttgttttttttgtggctgtggacatctttcaacactgccttcttggccttgcaaagccttcgctttggcttcggctttaggaggggcaggagcttccttcttcgttcttggcaccatcttatgaaaagggtccagattaagatttttgactgagtcattctaaagtaagttgcaagacccatgatactagaccactaaatacttcatcacacacctcctaagaataagaaccaacattatcacaccaaagaaaataaataattccataatatta

SEQ ID NO: 26 - DNA, 1301 bp, Homo sapiens
  HLA-F2
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 27 - DNA, 912 bp, Homo sapiens
  HLA-F3
atatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttataga

SEQ ID NO: 28 - DNA, 1578 bp, Homo sapiens
  HLA-G1
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 29 - DNA, 1302 bp, Homo sapiens
  HLA-G2
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 30 - DNA, 1026 bp, Homo sapiens
  HLA-G3
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 31 - DNA, 1302 bp, Homo sapiens
  HLA-G4
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 32 - DNA, 1138 bp, Homo sapiens
  HLA-G5
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 33 - DNA, 862 bp, Homo sapiens
  HLA-G6
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 34 - DNA, 529 bp, Homo sapiens
  HLA-G7
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagtgagtaa

SEQ ID NO: 35 - protein, 4 aa, Artificial Sequence
  redox-active site
CGPC

SEQ ID NO: 36 - DNA, 19 bp, Artificial Sequence
  HLA-G Ex3 Forward Primer
ggccggagtattgggaaga

SEQ ID NO: 37 - DNA, 23 bp, Artificial Sequence
  HLA-G Ex3 Probe
caaggcccacgcacagactgaca

SEQ ID NO: 38 - DNA, 20 bp, Artificial Sequence
  HLA-G Ex3 Reverse Primer
gcagggtctgcaggttcatt

SEQ ID NO: 39 - DNA, 19 bp, Artificial Sequence
  HLA-G Ex4 Forward Primer
ctgcggctcagatctccaa

SEQ ID NO: 40 - DNA, 21 bp, Artificial Sequence
  HLA-G Ex4 Probe
cgcaagtgtgaggcggccaat

SEQ ID NO: 41 - DNA, 23 bp, Artificial Sequence
  HLA-G Ex4 Reverse Primer
caggtaggctctcctttgttcag

SEQ ID NO: 42 - DNA, 24 bp, Artificial Sequence
  HLA-G Ex5 Forward Primer
caccaccctgtctttgactatgag

SEQ ID NO: 43 - DNA, 21 bp, Artificial Sequence
  HLA-G Ex5 Probe
accctgaggtgctgggccctg

SEQ ID NO: 44 - DNA, 24 bp, Artificial Sequence
  HLA-G Ex5 Reverse Primer
agtatgatctccgcagggtagaag

SEQ ID NO: 45 - DNA, 20 bp, Artificial Sequence
  HLA-G Ex6 Forward Primer
catccccatcatgggtatcg

SEQ ID NO: 46 - DNA, 22 bp, Artificial Sequence
  HLA-G Ex6 Probe
tgctggcctggttgtccttgca

SEQ ID NO: 47 - DNA, 20 bp, Artificial Sequence
  HLA-G Ex6 Reverse Primer
ccgcagctccagtgactaca

SEQ ID NO: 48 - DNA, 22 bp, Artificial Sequence
  HLA-G Ex8 Forward Primer
gaccctcttcctcatgctgaac

SEQ ID NO: 49 - DNA, 28 bp, Artificial Sequence
  HLA-G Ex8 Probe
cattccttccccaatcacctttcctgtt

SEQ ID NO: 50 - DNA, 19 bp, Artificial Sequence
  HLA-G Ex8 Reverse Primer
catcccagccccttttctg

SEQ ID NO: 51 - DNA, 19 bp, Artificial Sequence
  HLA-G Ex3-5 Forward Primer
ttcatcgccatgggctacg

SEQ ID NO: 52 - DNA, 22 bp, Artificial Sequence
  HLA-G Ex3-5 Probe
cgacacgcagttcgtgcggttc

SEQ ID NO: 53 - DNA, 19 bp, Artificial Sequence
  HLA-G Ex3-5 Reverse Primer
atcctcggacacgccgagt

SEQ ID NO: 54 - DNA, 20 bp, Artificial Sequence
  HLA-G Ex2/3 Forward Primer
ccgaaccctcttcctgctgc

SEQ ID NO: 55 - DNA, 20 bp, Artificial Sequence
  HLA-G Ex2/3 Probe
cgagacctgggcgggctccc

SEQ ID NO: 56 - DNA, 20 bp, Artificial Sequence
  HLA-G Ex2/3 Reverse Primer
gcgctgaaatacctcatgga

SEQ ID NO: 57 - DNA, 19 bp, Artificial Sequence
  HLA-H Ex2/3 Forward Primer
gagagaacctgcggatcgc

SEQ ID NO: 58 - DNA, 24 bp, Artificial Sequence
  HLA-H Ex2/3 Probe
agcgagggcggttctcacaccatg

SEQ ID NO: 59 - DNA, 19 bp, Artificial Sequence
  HLA-H Ex2/3 Reverse Primer
ccacgtcgcagccatacat

SEQ ID NO: 60 - DNA, 19 bp, Artificial Sequence
  HLA-H Forward Primer
gagagaacctgcggatcgc

SEQ ID NO: 61 - DNA, 25 bp, Artificial Sequence
  HLA-H Probe
accagagcgagggcggttctcacac

SEQ ID NO: 62 - DNA, 16 bp, Artificial Sequence
  HLA-H Reverse Primer
cgggccgggacatggt

SEQ ID NO: 63 - DNA, 18 bp, Artificial Sequence
  KRT5 Forward Primer
cgccacttaccgcaagct

SEQ ID NO: 64 - DNA, 24 bp, Artificial Sequence
  KRT5 Probe
tggagggcgaggaatgcagactca

SEQ ID NO: 65 - DNA, 25 bp, Artificial Sequence
  KRT5 Reverse Primer
acagagatgttgactggtccaactc

SEQ ID NO: 66 - DNA, 26 bp, Artificial Sequence
  KRT20 Forward Primer
gcgactacagtgcatattacagacaa

SEQ ID NO: 67 - DNA, 32 bp, Artificial Sequence
  KRT20 Probe
ttgaagagctgcgaagtcagattaaggatgct

SEQ ID NO: 68 - DNA, 21 bp, Artificial Sequence
  KRT20 Reverse Primer
cacaccgagcattttgcagtt

SEQ ID NO: 69 - DNA, 20 bp, Artificial Sequence
  CALM2 Forward Primer
gagcgagctgagtggttgtg

SEQ ID NO: 70 - DNA, 22 bp, Artificial Sequence
  CALM2 Probe
tcgcgtctcggaaaccggtagc

SEQ ID NO: 71 - DNA, 21 bp, Artificial Sequence
  CALM2 Reverse Primer
agtcagttggtcagccatgct

SEQ ID NO: 72 - DNA, 21 bp, Artificial Sequence
  HLA-L Ex2/3 Forward Primer
cctgctccgctattacaacca

SEQ ID NO: 73 - DNA, 26 bp, Artificial Sequence
  HLA-L Ex2/3 Probe
cgaggccggtatgaacagttcgccta

SEQ ID NO: 74 - DNA, 20 bp, Artificial Sequence
  HLA-L Ex2/3 Reverse Primer
cgttcagggcgatgtaatcc

SEQ ID NO: 75 - DNA, 18 bp, Artificial Sequence
  HLA-L Ex5/6 Forward Primer
gctgtggttgctgctgcg

SEQ ID NO: 76 - DNA, 29 bp, Artificial Sequence
  HLA-L Ex5/6 Probe
agaaaagctcaggcagcaattgtgctcag

SEQ ID NO: 77 - DNA, 30 bp, Artificial Sequence
  HLA-L Ex5/6 Reverse Primer
catagtcctctttacaagtatcatgagatg

SEQ ID NO: 78 - DNA, 23 bp, Artificial Sequence
  HLA-L Ex7 Forward Primer
tcctcttctgctcagctctccta

SEQ ID NO: 79 - DNA, 35 bp, Artificial Sequence
  HLA-L Ex7 Probe
ctctcccttccctgagttgtagtaatcctagcact

SEQ ID NO: 80 - DNA, 27 bp, Artificial Sequence
  HLA-L Ex7 Reverse Primer
gctttatagatccatgagtttgcatta

SEQ ID NO: 81 - DNA, 17 bp, Artificial Sequence
  HLA-J Ex4/5 Forward Primer
caaggggctgcccaagc

SEQ ID NO: 82 - DNA, 30 bp, Artificial Sequence
  HLA-J Ex4/5 Probe
catcctgagatgggtcacacatttctggaa

SEQ ID NO: 83 - DNA, 27 bp, Artificial Sequence
  HLA-J Ex4/5 Reverse Primer
cctcctagtcttggaaccttgagaagt